(19) United States
(12) Patent Application Publication
(10) Pub. No.: US 2022/0227716 A1
(43) Pub. Date: Jul. 21, 2022

(54) BENZIMIDAZOLE AND HYDROGENATED CARBAZOLE DERIVATIVES AS GPX4 INHIBITORS

(71) Applicant: Ferro Therapeutics, Inc., Palo Alto, CA (US)

(72) Inventors: Anjali Pandey, Fremont, CA (US); Chun Jiang, Hillsborough, CA (US); Biswajit Kalita, Bangalore (IN)

(21) Appl. No.: 17/638,741

(22) PCT Filed: Aug. 26, 2020
(86) PCT No.: PCT/US2020/047989

§ 371 Date: Feb. 25, 2022

Related U.S. Application Data
(60) Provisional application No. 62/893,130, filed on Aug. 28, 2019.
(60) Provisional application No. 62/893,123, filed on Aug. 28, 2019.

(51) Int. Cl. C07D 235/30; A61K 45/06; A61K 31/5377; C07D 401/04; C07D 209/82; A61K 31/403; C07D 405/08; A61P 35/00; A61P 35/02

(52) U.S. Cl. CPC C07D 235/30; A61K 45/06; A61K 31/5377; C07D 401/04; A61P 35/02; A61K 31/403; C07D 405/08; A61P 35/00; C07D 209/82

(57) ABSTRACT

This present disclosure relates to compounds with ferroptosis inducing activity, a method of treating a subject with cancer with the compounds, and combination treatments with a second therapeutic agent.

### CROSS-REFERENCE TO RELATED APPLICATIONS

This application claims priority under 35 U.S.C. 119(e) to U.S. Provisional Application Ser. No. 62/893,123, filed on Aug. 28, 2019, and 62/893,130, filed on Aug. 28, 2019, which are incorporated herein by reference in their entireties.

### BACKGROUND

Glutathione peroxidase 4 (GPX4) can directly reduce phospholipid hydroperoxide. Depletion of GPX4 induces lipid peroxidation-dependent cell death. Cancer cells in a drug-induced, therapy-resistant state have an enhanced dependence on the lipid peroxidase activity of GPX4 to prevent undergoing ferroptotic cell death. Studies have shown that lipophilic antioxidants, such as ferrostatin, can rescue cells from GPX4 inhibition-induced ferroptosis. For instance, mesenchymal state GPX4-knockout cells can survive in the presence of ferrostatin, however, when the supply of ferrostatin is terminated, these cells undergo ferroptosis (see, e.g., Viswanathan et al., Nature 547:453-7, 2017). It has also been experimentally determined that that GPX4i can be rescued by blocking other components of the ferroptosis pathways, such as lipid ROS scavengers (Ferrostatin, Liproxstatin), lipoxygenase inhibitors, iron chelators and caspase inhibitors, which an apoptotic inhibitor does not rescue. These findings are suggestive of non-apoptotic, iron-dependent, oxidative cell death (i.e., ferroptosis). Accordingly, a GPX4 inhibitor can be useful to induce ferroptotic cancer cell death and thus treat cancer.

### SUMMARY

The present disclosure relates to compounds having ferroptosis inducing activity, and methods of using the compounds for the treatment of cancer. In certain embodiments, provided herein is a compound of Formula I:

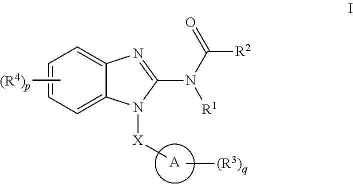

or a tautomer, stereoisomer, mixture of stereoisomers, isotopically enriched analog, or pharmaceutically acceptable salt thereof, wherein:

ring A is C₄-C₁₀cycloalkyl, heterocyclyl, aryl, or heteroaryl;

X is a covalent bond or —C(R⁹)₂—;

p is 0, 1, 2 or 3;

q is 0, 1, 2 or 3;

R¹ is hydrogen or C₁-C₆alkyl;

R² is —C₁-C₂haloalkyl optionally substituted with one or two —CH₃;

each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;

each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

each R⁹ is independently hydrogen or C₁-C₆alkyl;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, or —C₂-C₆alkenylheteroaryl.

In certain embodiments, provided herein is a compound of Formula A-I:

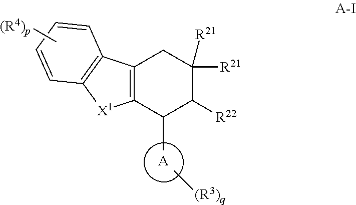

or a tautomer, stereoisomer, mixture of stereoisomers, isotopically enriched analog, or pharmaceutically acceptable salt thereof, wherein:

ring A is C₄-C₁₀cycloalkyl, heterocyclyl, aryl, or heteroaryl;

X is NR⁵, O or S;

p is 0, 1, 2 or 3;

q is 0, 1, 2 or 3;

each R²¹ is independently hydrogen, C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R)₂, —NO₂, —OR⁸, —C₁-C₆alkyl-OH, —C₁-C₆alkyl-OR⁸, or —Si(R⁵)₃;

R²² is —CN, —C(O)H, —C(O)OH, ethyleneoxide, —C(O)-ethyleneoxide, —C(O)—C₁-C₂alkyl, —C(O)—C₁-C₂haloalkyl, —C(O)—C₂-C₃alkenyl, —C(O)—C₂alkynyl, —NHC(O)—C₁-C₂haloalkyl, —NHC(O)—C₂-C₃alkenyl, —NHC(O)—C₂alkynyl, —CH(OH)—C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;

each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHRN(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;

each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;

R⁵ is hydrogen or C₁-C₆alkyl;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, or —C₂-C₆alkenylheteroaryl.

In certain embodiments, the compounds exhibit GPX4 inhibiting activity, and in certain embodiments, exhibit altered or enhanced stability (e.g., metabolic stability) and/or enhanced activity or other characteristics as compared to other GPX4 inhibitors. In certain embodiments, the compounds described herein are selective for GPX4 over other GPXs. In certain embodiments, the compounds are used in a method of inhibiting GPX4 in a cell, comprising contacting a cell with an effective amount of the compound described herein to inhibit GPX4 in the cell. In certain embodiments, the cell is a cancer cell.

In certain embodiments, provided is a method of inducing ferroptosis in a cell comprising contacting the cell with an effective amount of a compound or composition provided herein.

In certain embodiments, provided is a method for treating a cancer in a patient in need thereof, comprising administering an effective amount of a compound or composition provided herein. In certain embodiments, provided is a method for treating a malignant solid tumor in a patient in need thereof, comprising administering an effective amount of a compound or composition provided herein to the patient. In certain embodiments, the malignant solid tumor is a sarcoma, carcinoma, or lymphoma.

### DETAILED DESCRIPTION

As used in this specification and the appended claims, the singular forms “a,” “an” and “the” include plural referents unless the context clearly indicates otherwise. Thus, for example, reference to “a protein” includes more than one protein, and reference to “a compound” refers to more than one compound.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

It is to be understood that both the foregoing general description, including the drawings, and the following detailed description are exemplary and explanatory only and are not restrictive of this disclosure. The section headings used herein are for organizational purposes only and not to be construed as limiting the subject matter described.

### 1. Definitions

In reference to the present disclosure, the technical and scientific terms used in the descriptions herein will have the meanings commonly understood by one of ordinary skill in the art, unless specifically defined otherwise. Accordingly, the following terms are intended to have the meanings as described below.

“Ferroptosis” refers to a form of cell death understood in the art as involving generation of reactive oxygen species mediated by iron, and characterized by, in part, lipid peroxidation.

“Ferroptosis inducer” or “ferroptosis activator” refers to an agent which induces, promotes or activates ferroptosis.

“GPX4 inhibitor” refers to any agent that inhibits the activity of the enzyme glutathione peroxidase 4 (GPX4). A GPX4 inhibitor can be either a direct or indirect inhibitor. GPX4 is a phospholipid hydroperoxidase that in catalyzing the reduction of hydrogen peroxide and organic peroxides, thereby protects cells against membrane lipid peroxidation, or oxidative stress. GPX4 has a selenocysteine in the active site that is oxidized to a selenenic acid by the peroxide to afford a lipid-alcohol. The glutathione acts to reduce the selenenic acid (—SeOH) back to the selenol (—SeH). Should this catalytic cycle be disrupted, cell death occurs through an intracellular iron-mediated process known as ferroptosis.

“Subject” as used herein refers to a mammal, for example a dog, a cat, a horse, or a rabbit. In certain embodiments, the subject is a non-human primate, for example a monkey, chimpanzee, or gorilla. In certain embodiments, the subject is a human, sometimes referred to herein as a patient.

“Treating” or “treatment” of a disease, disorder, or syndrome, as used herein, includes (i) preventing the disease, disorder, or syndrome from occurring in a subject, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art, particularly in view of the guidance provided in the present disclosure.

“Therapeutically effective amount” refers to that amount which, when administered to an animal (e.g., human) for treating a disease, is sufficient to effect such treatment for the disease, disorder, or condition. In certain embodiments, the treatment provides a therapeutic benefit such as amelioration of symptoms or slowing of disease progression. For example, a therapeutically effective amount may be an amount sufficient to decrease a symptom of a disease or condition of as described herein.

The use of a dash, in certain embodiments, refers to a point of attachment. By way of example only, cycloalkylalkenyl- means that the point of attachment for a cycloalkylalkenyl substituent is the alkylene moiety.

“Alkyl” refers to a straight or branched chain hydrocarbon group of 1 to 20 carbon atoms (C₁-C₂₀ or C₁₋₂₀), e.g., 1 to 12 carbon atoms (C₁-C₁₂ or C₁₋₁₂), or 1 to 8 carbon atoms (C₁-C₈ or C₁₋₈). Exemplary “alkyl” includes, but are not limited to, methyl, ethyl, n-propyl, i-propyl, n-butyl, s-butyl, t-butyl, n-pentyl, and s-pentyl, and the like.

“Alkenyl” refers to a straight or branched chain hydrocarbon group of 2 to 20 carbon atoms (C₂-C₂₀ or C₂₋₂₀), e.g., 2 to 12 carbon atoms (C₂-C₁₂ or C₂₋₁₂), or 2 to 8 carbon atoms (C₂-C₈ or C₂₋₈), having at least one double bond. Exemplary “alkenyl” includes, but are not limited to, vinyl ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl, and the like.

“Alkynyl” refers to a straight or branched chain hydrocarbon group of 2 to 12 carbon atoms (C₂-C₁₂ or C₂₋₁₂), e.g., 2 to 8 carbon atoms (C₂-C₈ or C₂₋₈), containing at least one triple bond. Exemplary “alkynyl” includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl, and the like.

“Alkylene,” “alkenylene” and “alkynylene” refers to a straight or branched chain divalent hydrocarbon radical of the corresponding alkyl, alkenyl, and alkynyl, respectively. In certain embodiments, “alkyl,” “alkenyl,” and “alkynyl” can represent the corresponding “alkylene,” “alkenylene” and “alkynylene,” such as, by way of example and not limitation, cycloalkylalkyl-, heterocycloalkylalkyl-, arylalkyl-, heteroarylalkyl-, cycloalkylalkenyl-, heterocycloalkylalkenyl-, arylalkenyl-, heteroarylalkenyl-, cycloalkylalkynyl-, heterocycloalkylalkynyl-, arylalkynyl-, heteroarylalkynyl-, and the like, wherein the cycloalkyl, heterocycloalkyl, aryl, and heteroaryl group is connected, as a substituent via the corresponding alkylene, alkenylene, or alkynylene group.

“Lower” in reference to substituents refers to a group having between one and six carbon atoms.

“Alkylhalo” or “haloalkyl” refers to a straight or branched chain hydrocarbon group of 1 to 20 carbon atoms (C₁-C₂₀ or C₁₋₂₀), e.g., 1 to 12 carbon atoms (C₁-C₁₂ or C₁-12), or 1 to 8 carbon atoms (C₁-C₈ or C₁₋₈) wherein one or more (e.g., one to three, or one) hydrogen atom is replaced by a halogen (e.g., Cl, F, etc.). In certain embodiments, the term “alkylhalo” refers to an alkyl group as defined herein, wherein one hydrogen atom is replaced by a halogen (e.g., Cl, F, etc.). In certain embodiments, the term “alkylhalo” refers to an alkylchloride.

“Alkenylhalo” or “haloalkenyl” refers to a straight or branched chain hydrocarbon group of 2 to 20 carbon atoms (C₂-C₂₀ or C₂₋₂₀), e.g., 2 to 12 carbon atoms (C₂-C₁₂ or C₂₋₁₂), or 2 to 8 carbon atoms (C₂-C₈ or C₂₋₈), having at least one double bond, wherein one or more (e.g., one to three, or one) hydrogen atom is replaced by a halogen (e.g., Cl, F, etc.). In certain embodiments, the term “alkenylhalo” refers to an alkenyl group as defined herein, wherein one hydrogen atom is replaced by a halogen (e.g., Cl, F, etc.). In certain embodiments, the term “alkenylhalo” refers to an alkenylchloride.

“Cycloalkyl” refers to any stable monocyclic or polycyclic system which consists of carbon atoms, any ring of which being saturated. “Cycloalkenyl” refers to any stable monocyclic or polycyclic system which consists of carbon atoms, with at least one ring thereof being partially unsaturated. Examples of cycloalkyls include, but are not limited to, cyclopropyl, cyclobutyl, cyclopentyl, cyclohexyl, cycloheptyl, cyclooctyl, bicycloalkyls and tricycloalkyls (e.g., adamantyl).

“Heterocycloalkyl” or “heterocyclyl” refers to a 4 to 14 membered, mono- or polycyclic (e.g., bicyclic), non-aromatic hydrocarbon ring, wherein 1 to 3 carbon atoms are replaced by a heteroatom. Heteroatoms and/or heteroatomic groups which can replace the carbon atoms include, but are not limited to, —O—, —S—, —S—O—, —NR⁴⁰—, —PH—, —C(O)—, —S(O)—, —S(O)₂—, —S(O)NR⁴⁰—, —S(O)₂NR⁴⁰—, and the like, including combinations thereof, where each R⁴⁰ is independently hydrogen or lower alkyl. Examples include thiazolidinyl, thiadiazolyl, triazinyl, morpholinyl, pyrrolidinonyl, pyrrolidinyl, piperidinyl, piperazinyl, 2,3-dihydrofuranyl, dihydropyranyl, hydantoinyl, valerolactamyl, oxiranyl, oxetanyl, tetrahydrofuranyl, tetrahydropyranyl, dihydropyridinyl, tetrahydropyridinyl, tetrahydropyrimidinyl, tetrahydrothiophenyl, tetrahydrothiopyranyl, and the like. In certain embodiments, the “heterocycloalkyl” or “heterocyclyl” is a substituted or unsubstituted 4 to 7 membered monocyclic ring, wherein 1 to 3 carbon atoms are replaced by a heteroatom as described above.

In certain embodiments, the “heterocycloalkyl” or “heterocyclyl” is a 4 to 10, or 4 to 9, or 5 to 9, or 5 to 7, or 5 to 6 membered mono- or polycyclic (e.g., bicyclic) ring, wherein 1 to 3 carbon atoms are replaced by a heteroatom as described above. In certain embodiments, when the “heterocycloalkyl” or “heterocyclyl” is a substituted or unsubstituted bicyclic ring, one ring may be aromatic, provided at least one ring is non-aromatic, regardless of the point of attachment to the remainder of the molecule (e.g., indolinyl, isoindolinyl, and the like).

“Aryl” refers to a 6 to 14-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Examples of “aryl” groups include phenyl, naphthyl, indenyl, biphenyl, phenanthrenyl, naphthacenyl, and the like.

“Heteroaryl” means an aromatic heterocyclic ring, including monocyclic and polycyclic (e.g., bicyclic) ring systems, where at least one carbon atom of one or both of the rings is replaced with a heteroatom independently selected from nitrogen, oxygen, and sulfur, or at least two carbon atoms of one or both of the rings are replaced with a heteroatom independently selected from nitrogen, oxygen, and sulfur. In certain embodiments, the heteroaryl can be a 5 to 6 membered monocyclic, or 7 to 11 membered bicyclic ring systems. Examples of “heteroaryl” groups include pyrrolyl, pyrazolyl, imidazolyl, pyrazinyl, oxazolyl, isoxazolyl, thiazolyl, furyl, thienyl, pyridyl, pyrimidyl, benzothiazolyl, purinyl, benzimidazolyl, indolyl, isoquinolyl, quinoxalinyl, quinolyl, and the like.

“Bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 5-12 ring members and 0-4 heteroatoms independently selected from nitrogen, oxygen, and sulfur. Such bridged bicyclic groups include those groups set forth below where each group is attached to the rest of the molecule at any substitutable carbon or nitrogen atom. Exemplary bridged bicyclics include, but are not limited to:

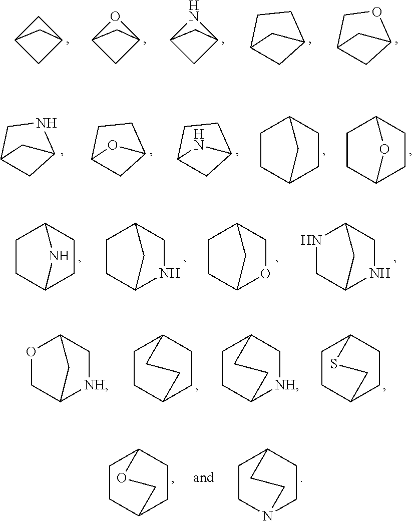

“Fused ring” refers a ring system with two or more rings having at least one bond and two atoms in common. A “fused aryl” and a “fused heteroaryl” refer to ring systems having at least one aryl and heteroaryl, respectively, that share at least one bond and two atoms in common with another ring.

“Halogen” or “halo” refers to fluorine, chlorine, bromine and iodine.

“Acyl” refers to —C(O)R⁴³, where R⁴³ is hydrogen, or an optionally substituted alkyl, heteroalkyl, cycloalkyl, cycloalkylalkyl, heterocycloalkyl, heterocycloalkylalkyl, aryl, arylalkyl, heteroaryl, or heteroarylalkyl as defined herein. Exemplary acyl groups include, but are not limited to, formyl, acetyl, cyclohexylcarbonyl, cyclohexylmethylcarbonyl, benzoyl, benzylcarbonyl, and the like.

“Alkyloxy” or “alkoxy” refers to —OR⁴⁴, wherein R⁴⁴ is an optionally substituted alkyl.

“Aryloxy” refers to —OR⁴⁵, wherein R⁴⁵ is an optionally substituted aryl.

“Carboxy” refers to —COO⁻ or COOM, wherein M is H or a counterion (e.g., a cation, such as Na⁺, Ca²⁺, Mg²⁺, etc.).

“Carbamoyl” refers to —C(O)NR⁴⁶R⁴⁶, wherein each R⁴⁶ is independently selected from H or an optionally substituted alkyl, cycloalkyl, cycloalkylalkyl, heterocycloalkyl, heterocylcoalkylalkyl, aryl, arylalkyl, heteroaryl, or heteroarylalkyl.

“Ester” refers to a group such as —C(═O)OR⁴⁷, alternatively illustrated as —C(O)OR⁴⁷, wherein R⁴⁷ is selected from an optionally substituted alkyl, cycloalkyl, cycloalkylalkyl, heterocycloalkyl, heterocyclolalkylalkyl, aryl, arylalkyl, heteroaryl, and heteroarylalkyl.

“Ether” refers to the group -alkyl-O-alkyl, where the term alkyl is as defined herein.

“Sulfanyl” refers to —SR⁴⁸, wherein R⁴⁸ is selected from an optionally substituted alkyl, cycloalkyl, cycloalkylalkyl, heterocycloalkyl, heterocycloalkylalkyl, aryl, arylalkyl, heteroaryl, and heteroarylalkyl. For example, —SR⁴⁸, wherein R⁴⁸ is an alkyl is an alkylsulfanyl.

“Sulfonyl” refers to —S(O)₂—, which may have various substituents to form different sulfonyl groups including sulfonic acids, sulfonamides, sulfonate esters, and sulfones. For example, —S(O)₂R⁴⁹, wherein R⁴⁹ is an alkyl refers to an alkylsulfonyl. In certain embodiments of —S(O)₂R⁴⁹, R⁴⁹ is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Sulfinyl” refers to —S(O)—, which may have various substituents to form different sulfinyl groups including sulfinic acids, sulfinamides, and sulfinyl esters. For example, —S(O)R⁵⁰, wherein R⁵⁰ is an alkyl refers to an alkylsulfinyl. In certain embodiments of —S(O)R⁵⁰, R⁵⁰ is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Silyl” refers to Si, which may have various substituents, for example —SiR⁵¹R⁵¹R⁵¹, where each R⁵¹ is independently selected from alkyl, cycloalkyl, cycloalkylalkyl, heterocycloalkyl, heterocycloalkylalkyl, aryl, arylalkyl, heteroaryl, and heteroarylalkyl. As defined herein, any heterocycloalkyl or heteroaryl group present in a silyl group has from 1 to 3 heteroatoms selected independently from O, N, and S.

“Amino” or “amine” refers to the group —NR⁵²R⁵² or —N⁺R⁵²R⁵²R⁵², wherein each R⁵² is independently selected from hydrogen and an optionally substituted alkyl, cycloalkyl, heterocycloalkyl, alkyloxy, aryl, heteroaryl, heteroarylalkyl, acyl, —C(O)—O-alkyl, sulfanyl, sulfinyl, sulfonyl, and the like. Exemplary amino groups include, but are not limited to, dimethylamino, diethylamino, trimethylammonium, triethylammonium, methylysulfonylamino, furanyl-oxy-sulfamino, and the like.

“Amide” refers to a group such as —C(═O)NR⁵³R⁵³, wherein each R⁵³ is independently selected from H and an optionally substituted alkyl, cycloalkyl, cycloalkylalkyl, heterocycloalkyl, heterocycloalkylalkyl, aryl, arylalkyl, heteroaryl, and heteroarylalkyl.

“Carbamate” refers to a group such as —O—C(═O)NR⁵³R⁵³ or —NR⁵³—C(═O)OR⁵³, wherein each R⁵³ is independently selected from H and an optionally substituted alkyl, cycloalkyl, cycloalkylalkyl, heterocycloalkyl, heterocycloalkylalkyl, aryl, arylalkyl, heteroaryl, and heteroarylalkyl.

“Sulfonamide” refers to —S(O)₂NR⁵⁴R⁵⁴, wherein each R⁵⁴ is independently selected from H and an optionally substituted alkyl, heteroalkyl, heteroaryl, heterocycle, alkenyl, alkynyl, arylalkyl, heteroarylalkyl, heterocyclylalkyl, alkylene-C(O)—OR⁵⁵, or alkylene-O—C(O)—OR⁵⁵, where R⁵⁵ is selected from H, alkyl, heteroalkyl, cycloalkyl, heterocyclyl, aryl, heteroaryl, alkenyl, alkynyl, arylalkyl, heterocycloalkyl, heteroarylalkyl, amino, and sulfinyl.

“Adamantyl” refers to a compound of structural formula:

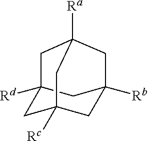

where optional substitutions can be present on one or more of R^{a}, R^{b}, R^{c}, and R^{d}. Adamantyl includes substituted adamantyl, e.g., 1- or 2-adamantyl, substituted by one or more substituents, including alkyl, halo, —OH, —NH₂, and alkoxy. Exemplary derivatives include methyladamatane, haloadamantane, hydroxyadamantane, and aminoadamantane (e.g., amantadine).

“N-protecting group” as used herein refers to those groups intended to protect a nitrogen atom against undesirable reactions during synthetic procedures. Exemplary N-protecting groups include, but is not limited to, acyl groups such acetyl and t-butylacetyl, pivaloyl, alkoxycarbonyl groups such as methyloxycarbonyl and t-butyloxycarbonyl (Boc), aryloxycarbonyl groups such as benzyloxycarbonyl (Cbz) and fluorenylmethoxycarbonyl (Fmoc and aroyl groups such as benzoyl. N-protecting groups are described in Greene's Protective Groups in Organic Synthesis, 5th Edition, P. G. M. Wuts, ed., Wiley (2014).

“Optional” or “optionally” refers to a described event or circumstance may or may not occur, and that the description includes instances where the event or circumstance occurs and instances where the event or circumstance does not. For example, “optionally substituted alkyl” refers to an alkyl group that may or may not be substituted and that the description encompasses both substituted alkyl group and unsubstituted alkyl group.

“Substituted” as used herein means one or more hydrogen atoms of the group is replaced with a substituent atom or group commonly used in pharmaceutical chemistry. Each substituent can be the same or different. Examples of suitable substituents include, but are not limited to, alkyl, alkenyl, alkynyl, cycloalkyl, aryl, arylalkyl, heterocycloalkyl, heteroaryl, —OR⁵⁶ (e.g., hydroxyl, alkyloxy (e.g., methoxy, ethoxy, and propoxy), ether, ester, carbamate, etc.), hydroxyalkyl, —C(O)O-alkyl, —O-alkyl-O-alkyl, haloalkyl, alkyl-O-alkyl, SR⁵⁶ (e.g., —SH, —S-alkyl, —S-aryl, —S-heteroaryl, arylalkyl-S—, etc.), S⁺R⁵⁶₂, S(O)R⁵⁶, SO₂R⁵⁶, NR⁵⁶R⁵⁷ (e.g., primary amine (i.e., NH₂), secondary amine, tertiary amine, amide, carbamate, urea, etc.), hydrazide, halo, nitrile, nitro, sulfide, sulfoxide, sulfone, sulfonamide, —SH, carboxy, aldehyde, keto, carboxylic acid, ester, amide, imine, and imide, including seleno and thio derivatives thereof, wherein each R⁵⁶ and R⁵⁷ are independently alkyl, alkenyl, alkynyl, heteroalkyl, cycloalkyl, cycloalkylalkyl, heterocycloalkyl, heterocycloalkylalkyl, aryl, arylalkyl, heteroaryl, or heteroarylalkyl, and wherein each of the substituents can be optionally further substituted. In embodiments in which a functional group with an aromatic carbon ring is substituted, such substitutions will typically number less than about 10 substitutions, or about 1 to 5, with about 1 or 2 substitutions in certain embodiments.

“Pharmaceutically acceptable salt” is meant to include salts of the active compounds which are prepared with relatively nontoxic acids or bases, depending on the particular substituents found on the compounds described herein. When compounds as disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds as disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure may contain both basic and acidic functionalities that allow the compounds to be converted into either base or acid addition salts. Lists of suitable salts are found in Remington's Pharmaceutical Sciences, 17th Ed., Mack Publishing Company, Easton, Pa., (1985) and Journal of Pharmaceutical Science, 66:2 (1977), each of which is incorporated herein by reference in its entirety.

“Pharmaceutically acceptable carrier” or “pharmaceutically acceptable excipient” refers to an excipient, carrier or adjuvant that can be administered to a subject, together with at least one compound, and which does not destroy the pharmacological activity thereof and is generally safe, nontoxic and neither biologically nor otherwise undesirable when administered in doses sufficient to deliver a therapeutic amount of the agent.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, e.g., a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds as disclosed herein, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

### 2. Compounds

In certain embodiments, provided herein is a compound of Formula I or a tautomer, stereoisomer, mixture of stereoisomers, isotopically enriched analog, or pharmaceutically acceptable salt thereof:

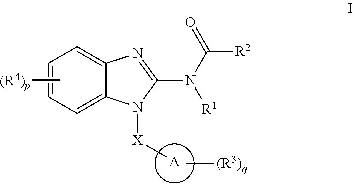

wherein:

ring A is C₄-C₁₀cycloalkyl, heterocyclyl, aryl, or heteroaryl;

X is a covalent bond or —C(R⁹)₂—;

p is 0, 1, 2 or 3;

q is 0, 1, 2 or 3;

R¹ is hydrogen or C₁-C₆alkyl;

R² is —C₁-C₂haloalkyl optionally substituted with one or two —CH₃;

each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;

each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

each R⁹ is independently hydrogen or C₁-C₆alkyl;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, or —C₂-C₆alkenylheteroaryl.

Also provided is a compound of Formula IA, or a tautomer, stereoisomer, mixture of stereoisomers, isotopically enriched analog, or pharmaceutically acceptable salt thereof:

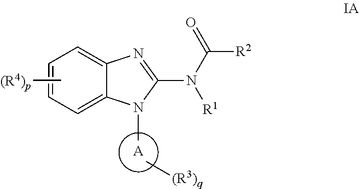

wherein each of ring A, R¹, R², R³, R⁴, p, and q are independently as defined herein.

Also provided is a compound of Formula IB, or a tautomer, stereoisomer, mixture of stereoisomers, isotopically enriched analog, or pharmaceutically acceptable salt thereof:

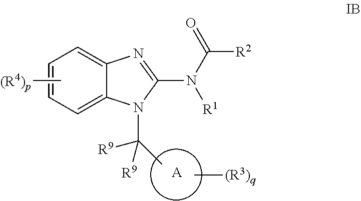

wherein each of ring A, R¹, R², R³, R⁴, R⁹, p, and q are independently as defined herein.

Also provided is a compound of Formula IC, or a tautomer, stereoisomer, mixture of stereoisomers, isotopically enriched analog, or pharmaceutically acceptable salt thereof:

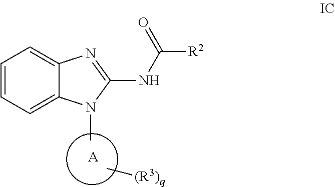

wherein each of ring A, R², R³, and q are independently as defined herein.

Also provided is a compound of Formula ID, or a tautomer, stereoisomer, mixture of stereoisomers, isotopically enriched analog, or pharmaceutically acceptable salt thereof:

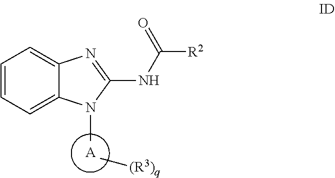

wherein each of ring A, R², R³, and q are independently as defined herein.

In certain embodiments, provided herein is a compound of Formula A-I or a tautomer, stereoisomer, mixture of stereoisomers, isotopically enriched analog, or pharmaceutically acceptable salt thereof:

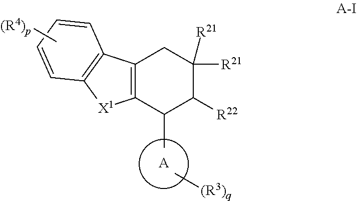

or a tautomer, stereoisomer, mixture of stereoisomers, isotopically enriched analog, or pharmaceutically acceptable salt thereof, wherein:

ring A is C₄-C₁₀cycloalkyl, heterocyclyl, aryl, or heteroaryl;

X¹ is NR⁵, O or S;

p is 0, 1, 2 or 3;

q is 0, 1, 2 or 3;

each R²¹ is independently hydrogen, C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R)₂, —NO₂, —OR⁸, —C₁-C₆alkyl-OH, —C₁-C₆alkyl-OR⁸, or —Si(R⁵)₃;

R²² is —CN, —C(O)H, —C(O)OH, ethyleneoxide, —C(O)-ethyleneoxide, —C(O)—C₁-C₂alkyl, —C(O)—C₁-C₂haloalkyl, —C(O)—C₂-C₃alkenyl, —C(O)—C₂alkynyl, —NHC(O)—C₁-C₂haloalkyl, —NHC(O)—C₂-C₃alkenyl, —NHC(O)—C₂alkynyl, —CH(OH)—C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;

each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;

each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;

R⁵ is hydrogen or C₁-C₆alkyl;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, or —C₂-C₆alkenylheteroaryl.

In certain embodiments, provided herein is a compound of Formula A-II, or a tautomer, stereoisomer, mixture of stereoisomers, isotopically enriched analog, or pharmaceutically acceptable salt thereof:

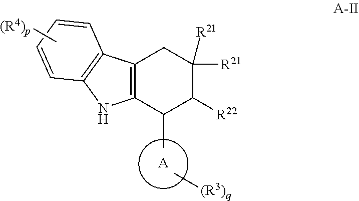

where A, R³, R⁴, R²¹, R²², p and q are as defined herein.

In certain embodiments, X¹ is NR⁵ or S.

In certain embodiments, R²² is —CN, —C(O)H, —C(O)OH, ethyleneoxide, —C(O)-ethyleneoxide, —C(O)—C₁-C₂alkyl, —C(O)—C₁-C₂haloalkyl, —C(O)—C₂-C₃alkenyl, —C(O)—C₂alkynyl, —NHC(O)—C₁-C₂haloalkyl, —NHC(O)—C₂-C₃alkenyl, or —NHC(O)—C₂alkynyl.

In certain embodiments, R²² is —CN, —C(O)—C₁-C₂alkyl, —C(O)—C₁-C₂haloalkyl, —C(O)—C₂-C₃alkenyl, —C(O)—C₂alkynyl, —NHC(O)—C₁-C₂haloalkyl, —NHC(O)—C₂-C₃alkenyl, or —NHC(O)—C₂alkynyl.

In certain embodiments, R²² is —C(O)C₁-C₂alkylhalo.

In certain embodiments, R²² is —C(O)CH₂Cl.

In certain embodiments, R²² is —C(O)C≡CH.

In certain embodiments, R²² is —CN.

In certain embodiments, when X¹ is NR⁵, then R² is —CN.

In certain embodiments, when X¹ is NR⁵, then R² is —C(O)—C₂alkynyl.

In certain embodiments, each R²¹ is independently C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸.

In certain embodiments, each R²¹ is independently C₁-C₆alkyl, C₂-C₆alkenyl, C₂-C₆alkynyl, C₁-C₆haloalkyl, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸.

In certain embodiments, each R²¹ is independently C₁-C₆alkyl, C₂-C₆alkenyl, C₂-C₆alkynyl, C₁-C₆haloalkyl, —CN, C₃-C₁₀cycloalkyl, —NH₂, —NHR⁸, —N(R⁸)₂, —OH, —OR⁸, —C₁-C₆alkyl-OH or —C₁-C₆alkyl-OR⁸.

In certain embodiments, each R²¹ is independently C₁-C₆alkyl, C₁-C₆haloalkyl, C₃-C₁₀cycloalkyl, —CN, —C(O)OR⁶, —C(O)N(R⁷)₂, —C₁-C₆alkyl-OH or —C₁-C₆alkyl-OR⁸.

In certain embodiments, each R²¹ is independently C₁-C₆alkyl, C₂-C₆alkenyl, C₂-C₆alkynyl, C₁-C₆haloalkyl, —NH₂, —NHR⁸, —N(R⁸)₂, —OH, —OR⁸, —C₁-C₆alkyl-OH or —C₁-C₆alkyl-OR⁸.

In certain embodiments, at least one R²¹ is independently C₁-C₆alkyl.

In certain embodiments, at least one R²¹ is independently C₃-C₁₀cycloalkyl.

In certain embodiments, each R²¹ is independently C₁-C₆alkyl. In certain embodiments, each R²¹ is methyl.

In certain embodiments, provided herein is a compound of Formula A-III, or a tautomer, stereoisomer, mixture of stereoisomers, isotopically enriched analog, or pharmaceutically acceptable salt thereof:

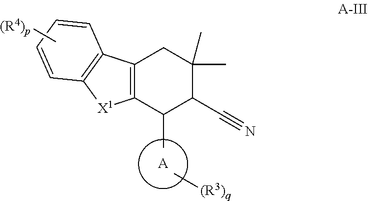

where A, R³, R⁴, p and q are as defined herein.

As used herein, “Formula I or sub-formulae thereof” refers to Formula I and/or Formula IA and/or Formula IB and/or Formula IC and/or Formula ID and/or Formula IE and/or Formula IF and/or Formula IG and/or Formula IH and/or Formula IIA and/or Formula JIB and/or Formula IIC and/or Formula IIIA and/or Formula IIIB and/or Formula IIIC.

As used herein, “Formula A-I or sub-formulae thereof” refers to Formula A-I and/or Formula A-II and/or Formula A-III. Unless specified otherwise, embodiments described herein refer to Formula I or sub-formulae thereof and/or Formula A-I or sub-formulae thereof.

In certain embodiments, ring A is:

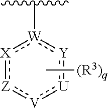

wherein 0 to 3 of U, V, W, X, Y, and Z is independently N, S, or O, and the remaining variables are CH or CR³ and each [symbol] independently represents a single or double bond, which comply with valency requirements based on U, V, W, X, Y and Z.

In certain embodiments, ring A is:

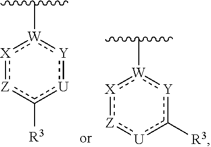

wherein 1 to 3 of U, W, X, Y, and Z is N, S, or O, and the remaining variables are CH or CR³ and [symbol] represents a single or double bond, which comply with valency requirements based on U, W, X, Y and Z.

In certain embodiments, ring A is aryl or heteroaryl. In certain embodiments, ring A is a monocyclic aryl or monocyclic heteroaryl.

In certain embodiments, ring A is aryl. In certain embodiments, ring A is phenyl.

In certain embodiments, ring A is heteroaryl. In certain embodiments, ring A is pyridyl. In certain embodiments, ring A is phenyl, pyridyl, piperidynyl, piperazinyl, or morpholinyl.

In certain embodiments, ring A is heterocyclyl. In certain embodiments, ring A is a 4 to 7 membered heterocyclyl.

In certain embodiments, ring A is aryl or heteroaryl, each of which is substituted by one to three R³. In certain embodiments, ring A is aryl or heteroaryl, each of which is substituted by one to three R³, where at least one R³ is C₃-C₁₀cycloalkyl, heterocyclyl, aryl, or heteroaryl; wherein each C₃-C₁₀cycloalkyl, heterocyclyl, aryl, and heteroaryl of R³ is optionally substituted with one to three R¹⁰.

In certain embodiments, ring A is aryl or heteroaryl, each of which is substituted with one, two or three R³. In certain embodiments, ring A is aryl or heteroaryl, each of which is substituted by two or three R³. In certain embodiments, ring A is aryl or heteroaryl, each of which is substituted with one, two or three R³. In certain embodiments, ring A is aryl or heteroaryl, each of which is substituted by two or three R³; wherein at least one R³ is halo.

In certain embodiments, ring A is cyclohexyl, substituted with one to three R³. In certain embodiments, ring A is C₄-C₁₀cycloalkyl, substituted with one two or three R³. In certain embodiments, ring A is a C₄-C₇cycloalkyl, substituted with one two or three R³. In certain embodiments, ring A is bicyclo[1.1.1]pentanyl, substituted with one two or three R³. In certain embodiments, ring A is selected from cyclobutyl, cyclopentyl, cyclohexyl, and cycloheptyl, wherein each is substituted with one two or three R³.

In certain embodiments, ring A is cyclohexyl. In certain embodiments, ring A is C₄-C₁₀cycloalkyl. In certain embodiments, ring A is a C₄-C₇cycloalkyl. In certain embodiments, ring A is bicyclo[1.1.1]pentanyl. In certain embodiments, ring A is selected from cyclobutyl, cyclopentyl, cyclohexyl, and cycloheptyl.

In certain embodiments, ring A is:

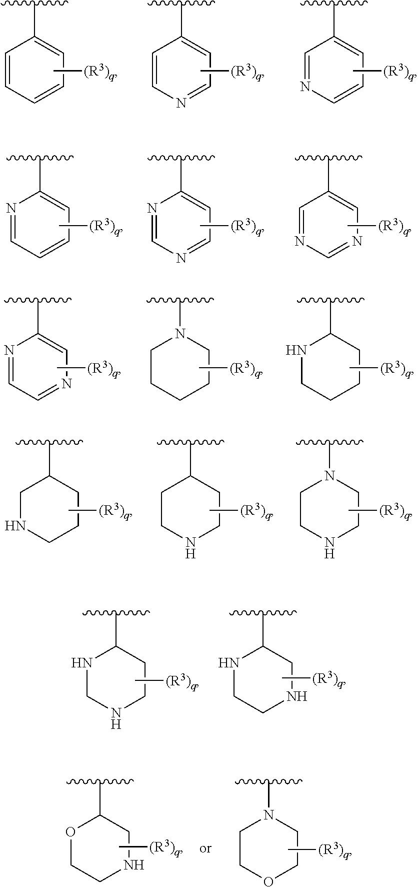

where q and each R³ is independently as defined herein.

In certain embodiments, ring A is:

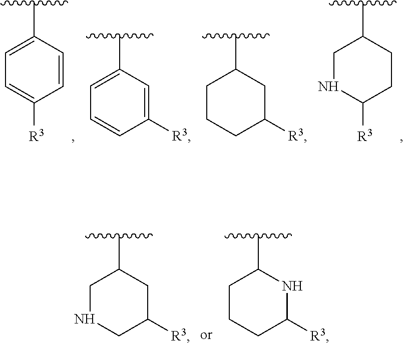

where R³ is independently as defined herein.

In certain embodiments, ring A is a bridged bicyclic ring selected from:

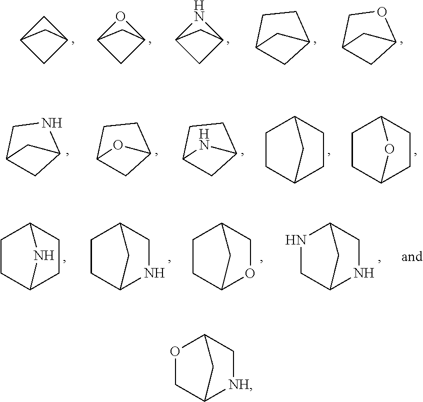

where each is substituted with one to three R³. In certain embodiments, ring A is a bridged bicyclic ring selected from:

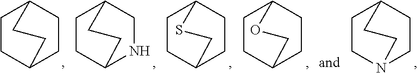

wherein each R³ is attached to a carbon atom on the bridged bicyclic ring.

In certain embodiments, ring A is:

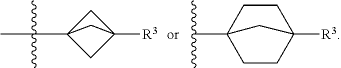

Also provided is a compound of Formula IE, or a tautomer, stereoisomer, mixture of stereoisomers, isotopically enriched analog, or pharmaceutically acceptable salt thereof:

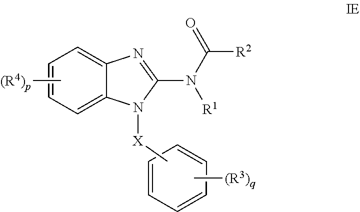

wherein each of X, R¹, R², R³, R⁴, p, and q are independently as defined herein.

Also provided is a compound of Formula IF, or a tautomer, stereoisomer, mixture of stereoisomers, isotopically enriched analog, or pharmaceutically acceptable salt thereof:

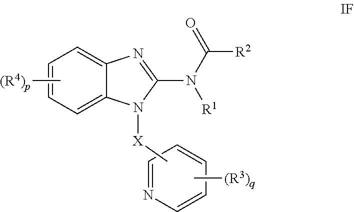

wherein each of X, R¹, R², R³, R⁴, p, and q are independently as defined herein.

Also provided is a compound of Formula IG, or a tautomer, stereoisomer, mixture of stereoisomers, isotopically enriched analog, or pharmaceutically acceptable salt thereof:

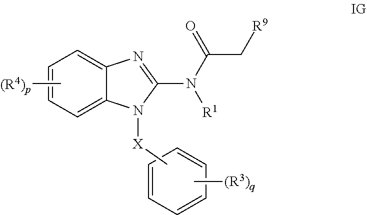

wherein each of X, R¹, R², R³, R⁴, p, and q are independently as defined herein, and R⁹ is halo.

Also provided is a compound of Formula IH, or a tautomer, stereoisomer, mixture of stereoisomers, isotopically enriched analog, or pharmaceutically acceptable salt thereof:

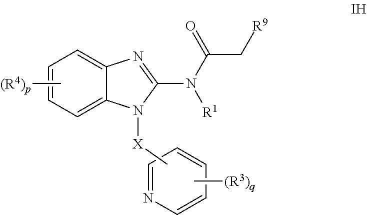

wherein each of X, R¹, R², R³, R⁴, p, and q are independently as defined herein, and R⁹ is halo.

Also provided is a compound of Formula IIA, or a tautomer, stereoisomer, mixture of stereoisomers, isotopically enriched analog, or pharmaceutically acceptable salt thereof:

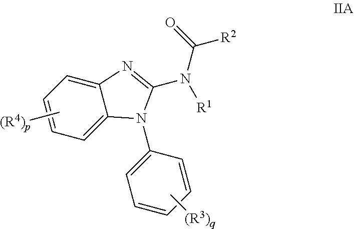

wherein each of R¹, R², R³, R⁴, p, and q are independently as defined herein.

Also provided is a compound of Formula IIB, or a tautomer, stereoisomer, mixture of stereoisomers, isotopically enriched analog, or pharmaceutically acceptable salt thereof:

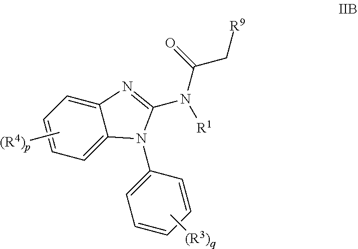

wherein each of R¹, R³, R⁴, p, and q are independently as defined herein, and R⁹ is halo.

Also provided is a compound of Formula IIIA, or a tautomer, stereoisomer, mixture of stereoisomers, isotopically enriched analog, or pharmaceutically acceptable salt thereof:

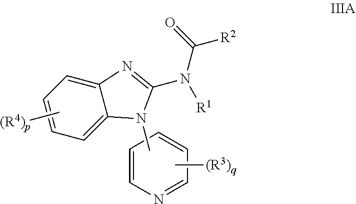

wherein each of R¹, R², R³, R⁴, p, and q are independently as defined herein.

Also provided is a compound of Formula IIIB, or a tautomer, stereoisomer, mixture of stereoisomers, isotopically enriched analog, or pharmaceutically acceptable salt thereof:

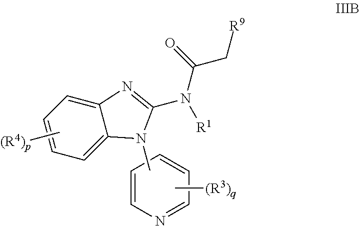

wherein each of R¹, R³, R⁴, p, and q are independently as defined herein, and R⁹ is halo.

Also provided is a compound of Formula IIC, or a tautomer, stereoisomer, mixture of stereoisomers, isotopically enriched analog, or pharmaceutically acceptable salt thereof:

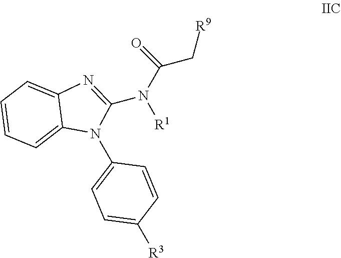

wherein each of R¹ and R³ are independently as defined herein, and R⁹ is halo.

Also provided is a compound of Formula IIIC, or a tautomer, stereoisomer, mixture of stereoisomers, isotopically enriched analog, or pharmaceutically acceptable salt thereof:

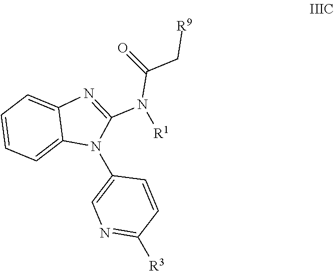

wherein each of R¹ and R³ are independently as defined herein, and R⁹ is halo.

In certain embodiments of Formula I or sub-formulae thereof, R¹ is C₁-C₆alkyl. In certain embodiments, R¹ is methyl. In certain embodiments, R¹ is hydrogen.

In certain embodiments of Formula I or sub-formulae thereof, ring A is aryl or heteroaryl;

X is a bond or —CH₂—;

p is 0, 1 or 2;

q is 1;

R¹ is hydrogen or methyl;

R³ is halo, —NHR⁸, —S(O)₂N(R⁷)₂, —C(O)OR⁶, —C(O)N(R⁷)₂, or heterocyclyl;

each R⁴ is independently halo or —OR⁸;

R⁶ is C₁-C₆alkyl;

each R⁷ is independently hydrogen, C₁-C₆alkyl, or C₃-C₁₀cycloalkyl, wherein each R⁷ is independently further substituted with one to three R¹¹;

each R⁸ is independently C₁-C₆alkyl or C₃-C₁₀cycloalkyl; wherein each R⁸ is independently further substituted with one to three R¹¹; and

each R¹¹ is independently —O—C₁-C₆alkyl.

In certain embodiments of Formula A-I or sub-formulae thereof, ring A is C₄-C₁₀cycloalkyl, heterocyclyl, aryl, or heteroaryl;

X¹ is NR⁵ or S;

p is 0, 1, 2 or 3;

q is 0, 1, 2 or 3;

each R²¹ is independently C₁-C₆alkyl, C₁-C₆haloalkyl, C₃-C₁₀cycloalkyl, —CN, —C(O)OR⁶, —C(O)N(R⁷)₂, —C₁-C₆alkyl-OH or —C₁-C₆alkyl-OR⁸;

R²² is —CN, —C(O)H, —C(O)OH, ethyleneoxide, —C(O)-ethyleneoxide, —C(O)—C₁-C₂alkyl, —C(O)—C₁-C₂haloalkyl, —C(O)—C₂-C₃alkenyl, —C(O)—C₂alkynyl, —NHC(O)—C₁-C₂haloalkyl, —NHC(O)—C₂-C₃alkenyl, or —NHC(O)—C₂alkynyl;

each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently optionally substituted with one to three R¹⁰;

each R⁴ is independently halo, —CN, —OH, —OR⁸, C₁-C₆alkyl, or C₂-C₆alkynyl; wherein the C₁-C₆alkyl of R⁴ is optionally independently optionally substituted with one to three R¹⁰;

R⁵ is hydrogen or C₁-C₆alkyl;

each R⁶ is independently hydrogen, C₁-C₆alkyl, C₂-C₆alkenyl, or —C₁-C₆alkylC₃-C₁₀cycloalkyl; wherein each R⁶ is independently further substituted with one to three R¹¹;

each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three R¹¹;

each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is optionally independently substituted with one to three R¹¹;

each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

In certain embodiments, at least one R³ is halo.

In certain embodiments, at least one R³ is —NHR⁸. In certain embodiments, at least one R³ is —N(R⁸)₂. In certain embodiments, q is 2, and one R³ is halo and the other R³ is —N(R⁸)₂. In certain embodiments, q is 3, and two R³ are independently halo and one R³ is —N(R⁸)₂.

In certain embodiments, at least one R³ is —C(O)OR⁶ or —C(O)R⁶.

In certain embodiments, at least one R³ is —S(O)₂N(R⁷)₂, —S(O)N(R⁷)₂, or —C(O)N(R⁷)₂.

In certain embodiments, at least one R³ is —S(O)₂R⁸, —S(O)R⁸, —NR¹²C(O)R⁸, —NR¹²C(O)OR⁸, —OC(O)R⁸, or —OC(O)CHR⁸N(R¹²)₂.

In certain embodiments, each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently optionally substituted with one to three R¹⁰.

In certain embodiments, each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl is independently optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments of Formula I or sub-formulae thereof, each R³ is independently halo, —NHR⁸, —C(O)N(R⁷)₂, or heterocyclyl.

In certain embodiments, q is 1, and R³ is —S(O)₂N(R⁷)₂, —S(O)N(R⁷)₂, or —C(O)N(R⁷)₂.

In certain embodiments, q is 1, and R³ is halo.

In certain embodiments, q is 1, and R³ is —C(O)N(R⁷)₂.

In certain embodiments, q is 1, and R³ is heterocyclyl.

In certain embodiments of Formula A-I or sub-formulae thereof, at least one R³ is —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

In certain embodiments of Formula A-I or sub-formulae thereof, at least one R³ is —NHR⁸ or —N(R⁸)₂.

In certain embodiments of Formula A-I or sub-formulae thereof, at least one R³ is —C(O)OR⁶ or —C(O)R⁶.

In certain embodiments of Formula A-I or sub-formulae thereof, at least one R³ is —S(O)₂N(R⁷)₂, —S(O)N(R⁷)₂, or —C(O)N(R⁷)₂.

In certain embodiments of Formula A-I or sub-formulae thereof, at least one R³ is —S(O)₂R⁸, —S(O)R⁸, —NR¹²C(O)R⁸, —NR¹²C(O)OR⁸, —OC(O)R⁸, or —OC(O)CHR⁸N(R¹²)₂.

In certain embodiments, each R⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently optionally substituted with one to three R¹⁰.

In certain embodiments, each R⁴ is independently halo, —CN, —OR⁸, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

In certain embodiments, each R⁴ is independently halo, —CN, —OH, —OR⁸, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

In certain embodiments, each R⁴ is independently halo, —CN, —OH, —OR⁸, C₁-C₆alkyl, or C₂-C₆alkynyl; wherein the C₁-C₆alkyl of R⁴ is optionally substituted with one to three R¹⁰.

In certain embodiments, each R⁴ is independently halo, —CN, —OH, —OR⁸, C₁-C₆alkyl, C₂-C₆alkynyl; wherein the C₁-C₆alkyl of R⁴ is optionally substituted with one to three substituents independently selected from —OR¹², —N(R²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments of Formula I or sub-formulae thereof, each R⁴ is independently halo, —CN, —OH, C₁-C₆alkyl, C₂-C₆alkynyl, or C₃-C₁₀cycloalkyl.

In certain embodiments of Formula I or sub-formulae thereof, each R⁴ is independently halo or —OR⁸.

In certain embodiments of Formula I or sub-formulae thereof, each R⁴ is independently halo —OH or —OCH₃.

In certain embodiments, each R⁶ is independently hydrogen, C₁-C₆alkyl, C₂-C₆alkenyl, or —C₁-C₆alkylC₃-C₁₀cycloalkyl; wherein each R⁶ is independently further substituted with one to three R¹¹.

In certain embodiments, each R⁶ is independently hydrogen, C₁-C₆alkyl, C₂-C₆alkenyl, or —C₁-C₆alkylC₃-C₁₀cycloalkyl; wherein each R⁶ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three R¹¹.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three R¹¹.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is optionally independently substituted with one to three R¹¹;

each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R¹⁵ is independently C₁-C₆alkyl.

In certain embodiments, p is 1, 2 or 3. In certain embodiments, p is 1. In certain embodiments, p is 2. In certain embodiments, p is 3. In certain embodiments, p is 0. In certain embodiments, p is 0 or 1.

In certain embodiments, p is 1 or 2. In certain embodiments, p is 0, 1 or 2.

In certain embodiments, q is 1, 2 or 3. In certain embodiments, q is 1. In certain embodiments, q is 2. In certain embodiments, q is 3. In certain embodiments, q is 0. In certain embodiments, q is 0 or 1. In certain embodiments, q is 1 or 2.

Also provided is a compound, or a tautomer, stereoisomer, mixture of stereoisomers, isotopically enriched analog, or pharmaceutically acceptable salt thereof, selected from Table 1:

TABLE 1

| No. | Structures |
|---|---|
| 1 | 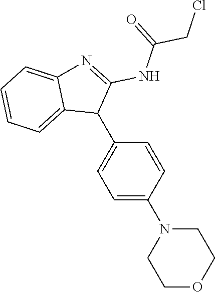 [SMILES: O=C(CCl)NC1=Nc2ccccc2C1c1ccc(N2CCOCC2)cc1] |
| 2 | 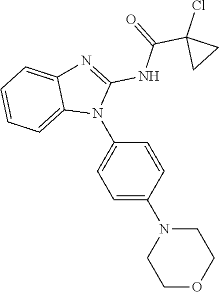 [SMILES: O=C(Nc1nc2ccccc2n1-c1ccc(N2CCOCC2)cc1)C1(Cl)CC1] |
| 3 | 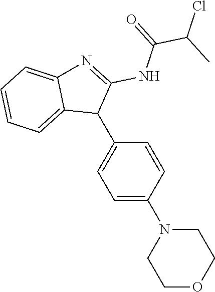 [SMILES: CC(Cl)C(=O)NC1=Nc2ccccc2C1c1ccc(N2CCOCC2)cc1] |
| 4 | 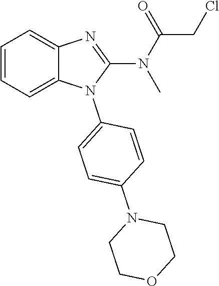 [SMILES: CN(C(=O)CCl)c1nc2ccccc2n1-c1ccc(N2CCOCC2)cc1] |
| 5 | 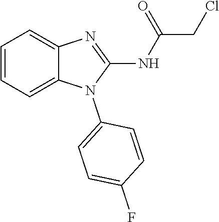 [SMILES: O=C(CCl)Nc1nc2ccccc2n1-c1ccc(F)cc1] |
| 6 | 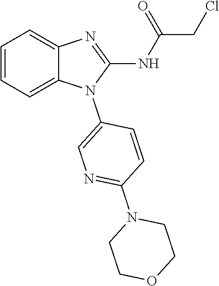 [SMILES: O=C(CCl)Nc1nc2ccccc2n1-c1ccc(N2CCOCC2)nc1] |
| 7 | 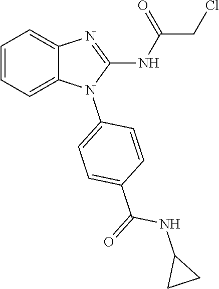 [SMILES: O=C(CCl)Nc1nc2ccccc2n1-c1ccc(C(=O)NC2CC2)cc1] |
| 8 | 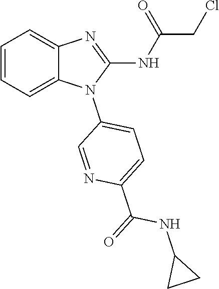 [SMILES: O=C(CCl)Nc1nc2ccccc2n1-c1ccc(C(=O)NC2CC2)nc1] |
| 9 | 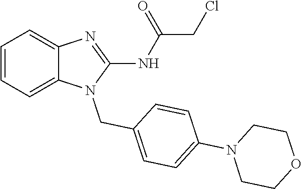 [SMILES: O=C(CCl)Nc1nc2ccccc2n1Cc1ccc(N2CCOCC2)cc1] |
| 10 | 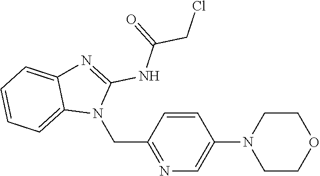 [SMILES: O=C(CCl)Nc1nc2ccccc2n1Cc1ccc(N2CCOCC2)cn1] |
| 11 | 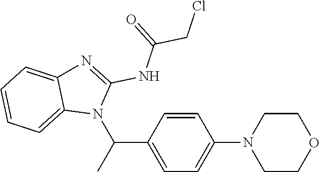 [SMILES: CC(c1ccc(N2CCOCC2)cc1)n1c(NC(=O)CCl)nc2ccccc21] |
| 12 | 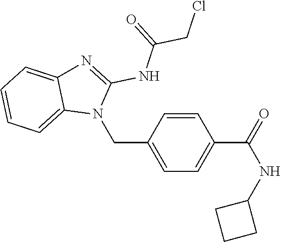 [SMILES: O=C(CCl)Nc1nc2ccccc2n1Cc1ccc(C(=O)NC2CCC2)cc1] |

Also provided is a compound, or a tautomer, stereoisomer, mixture of stereoisomers, isotopically enriched analog, or pharmaceutically acceptable salt thereof, selected from Table A-1:

TABLE A-1

| No. | Structure |
|---|---|
| A-1 | 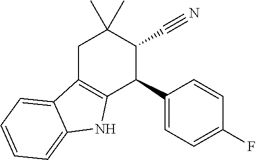 [SMILES: CC1(C)Cc2c([nH]c3ccccc23)[C@H](c2ccc(F)cc2)[C@H]1C#N] |
| A-2 | 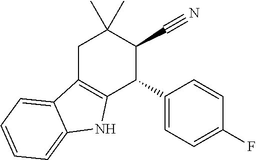 [SMILES: CC1(C)Cc2c([nH]c3ccccc23)[C@@H](c2ccc(F)cc2)[C@@H]1C#N] |
| A-3 | 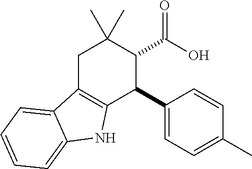 [SMILES: Cc1ccc([C@H]2c3[nH]c4ccccc4c3CC(C)(C)[C@@H]2C(=O)O)cc1] |
| A-4 | 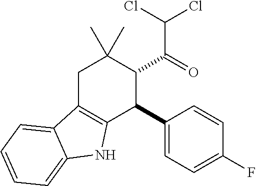 [SMILES: CC1(C)Cc2c([nH]c3ccccc23)[C@H](c2ccc(F)cc2)[C@H]1C(=O)C(Cl)Cl] |
| A-5 | 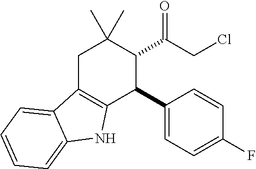 [SMILES: CC1(C)Cc2c([nH]c3ccccc23)[C@H](c2ccc(F)cc2)[C@H]1C(=O)CCl] |
| A-6 | 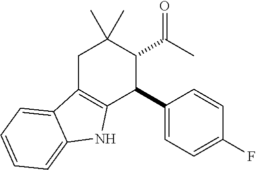 [SMILES: CC(=O)[C@@H]1[C@@H](c2ccc(F)cc2)c2[nH]c3ccccc3c2CC1(C)C] |
| A-7 | 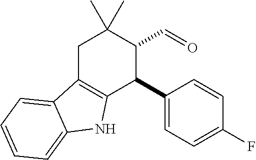 [SMILES: CC1(C)Cc2c([nH]c3ccccc23)[C@H](c2ccc(F)cc2)[C@H]1C=O] |
| A-8 | 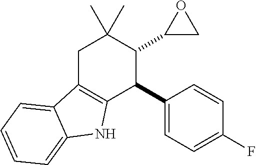 [SMILES: CC1(C)Cc2c([nH]c3ccccc23)[C@H](c2ccc(F)cc2)[C@H]1C1CO1] |
| A-9 | 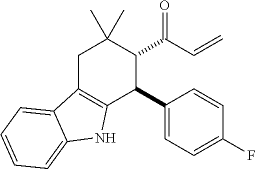 [SMILES: C=CC(=O)[C@@H]1[C@@H](c2ccc(F)cc2)c2[nH]c3ccccc3c2CC1(C)C] |
| A-10 | 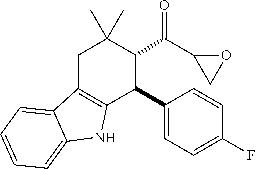 [SMILES: CC1(C)Cc2c([nH]c3ccccc23)[C@H](c2ccc(F)cc2)[C@H]1C(=O)C1CO1] |
| A-11 | 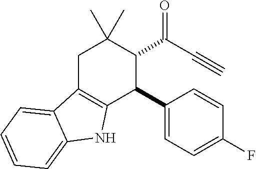 [SMILES: C#CC(=O)[C@@H]1[C@@H](c2ccc(F)cc2)c2[nH]c3ccccc3c2CC1(C)C] |
| A-12 | 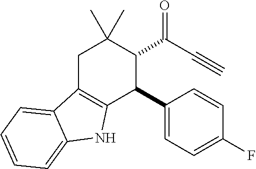 [SMILES: C#CC(=O)[C@@H]1[C@@H](c2ccc(F)cc2)c2[nH]c3ccccc3c2CC1(C)C] |
| A-13 | 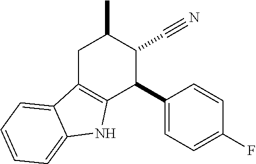 [SMILES: C[C@@H]1Cc2c([nH]c3ccccc23)[C@H](c2ccc(F)cc2)[C@H]1C#N] |
| A-14 | 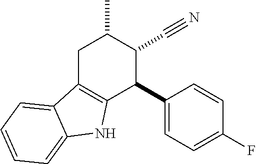 [SMILES: C[C@H]1Cc2c([nH]c3ccccc23)[C@H](c2ccc(F)cc2)[C@H]1C#N] |
| A-15 | 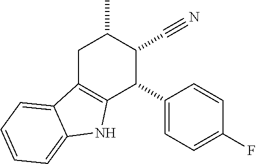 [SMILES: C[C@H]1Cc2c([nH]c3ccccc23)[C@@H](c2ccc(F)cc2)[C@H]1C#N] |
| A-16 | 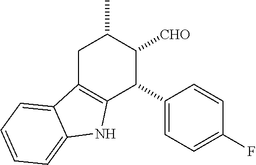 [SMILES: C[C@H]1Cc2c([nH]c3ccccc23)[C@@H](c2ccc(F)cc2)[C@H]1C=O] |
| A-17 | 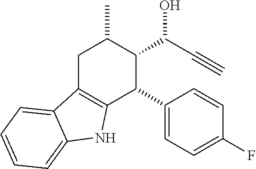 [SMILES: C#C[C@@H](O)[C@@H]1[C@H](c2ccc(F)cc2)c2[nH]c3ccccc3c2C[C@@H]1C] |
| A-18 | 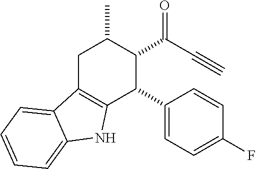 [SMILES: C#CC(=O)[C@@H]1[C@H](c2ccc(F)cc2)c2[nH]c3ccccc3c2C[C@@H]1C] |
| A-19 | 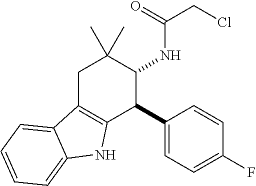 [SMILES: CC1(C)Cc2c([nH]c3ccccc23)[C@H](c2ccc(F)cc2)[C@H]1NC(=O)CCl] |

### 3. Methods of Use

In certain embodiments, the compounds described herein are used in a method of treating cancer. In certain embodiments, the method of treating cancer comprises administering to a subject in need thereof a therapeutically effective amount any of the compounds described herein.

In certain embodiments, the compounds are used in a method of inhibiting GPX4 in a cell, comprising contacting a cell with an effective amount of a compound or composition described herein to inhibit GPX4 in the cell. In certain embodiments, the cell is a cancer cell. In certain embodiments, the method comprises administering an effective amount of a compound or composition described herein to a patient in need thereof.

In certain embodiments, the compounds are used in a method of inducing ferroptosis in a cell comprising contacting the cell with an effective amount of a compound or composition provided herein.

In certain embodiments, the method comprises administering an effective amount of a compound or composition described herein to a patient in need thereof.

In certain embodiments, provided is a method for treating a cancer in a patient in need thereof, comprising administering an effective amount of a compound or composition provided herein.

In certain embodiments, the compounds are used in a method of treating cancer in a subject in need thereof, comprising administering to a subject having cancer a therapeutically effective amount of a ferroptosis inducing compound disclosed herein. Various cancers for treatment with the compounds include, but are not limited to, adrenocortical cancer, anal cancer, biliary cancer, bladder cancer, bone cancer, gliomas, astrocytoma, neuroblastoma, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, and soft tissue carcinomas. In certain embodiments, the compound is used to treat pancreatic cancer.

In certain embodiments, the cancer is renal cell carcinoma (RCC), pancreatic cancer, lung cancer, breast cancer, or prostate cancer. In certain embodiments, provided is a method for treating renal cell carcinoma (RCC) in a patient in need thereof, comprising administering an effective amount of a compound or composition provided herein. In certain embodiments, provided is a method for treating pancreatic cancer in a patient in need thereof, comprising administering an effective amount of a compound or composition provided herein. In certain embodiments, provided is a method for treating lung cancer in a patient in need thereof, comprising administering an effective amount of a compound or composition provided herein. In certain embodiments, provided is a method for treating breast cancer in a patient in need thereof, comprising administering an effective amount of a compound or composition provided herein. In certain embodiments, provided is a method for treating prostate cancer in a patient in need thereof, comprising administering an effective amount of a compound or composition provided herein.

In certain embodiments, provided is a method for treating a malignant solid tumor in a patient in need thereof, comprising administering an effective amount of a compound or composition provided herein to the patient. In certain embodiments, the malignant solid tumor is a carcinoma. In certain embodiments, the malignant solid tumor is a lymphoma. In certain embodiments, the malignant solid tumor is a sarcoma.

In certain embodiments, the cancer for treatment with the compound can be selected from, among others, adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemia and lymphoma), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, vaginal cancer, sarcoma, and soft tissue carcinomas. In certain embodiments, the cancer is renal cell carcinoma (RCC). In certain embodiments, the cancer is pancreatic cancer. In certain embodiments, the cancer is lung cancer. In certain embodiments, the cancer is breast cancer. In certain embodiments, the cancer is prostate cancer.

In certain embodiments, the cancer for treatment with the compound is pancreatic cancer. In certain embodiments, the pancreatic cancer for treatment with the compounds is pancreatic adenocarcinoma or metastatic pancreatic cancer. In certain embodiments, the cancer for treatment with the compounds is stage I, stage II, stage III, or stage IV pancreatic adenocarcinoma.

In certain embodiments, the cancer for treatment with the compounds is lung cancer. In certain embodiments, the lung cancer for treatment with the compounds is small cell lung cancer or non-small cell lung cancer. In certain embodiments, the non-small cell lung cancer for treatment with the compounds is an adenocarcinoma, squamous cell carcinoma, or large cell carcinoma. In certain embodiments, the lung cancer for treatment with the compounds is metastatic lung cancer.

In certain embodiments, the cancer for treatment with the compounds is a hematologic cancer. In certain embodiments, the hematologic cancer is selected from acute lymphoblastic leukemia (ALL), acute myeloid leukemia (AML), lymphoma (e.g., Hodgkin's lymphoma, Non-Hodgkin's lymphoma, Burkitt's lymphoma), chronic lymphocytic leukemia (CLL), chronic myelogenous leukemia (CML), Hairy Cell chronic myelogenous leukemia (CML), and multiple myeloma.

In certain embodiments, the cancer for treatment with the compounds is a leukemia selected from acute lymphoblastic leukemia (ALL), acute myeloid leukemia (AML), chronic lymphocytic leukemia (CLL), chronic myelogenous leukemia (CML), Hairy Cell chronic myelogenous leukemia (CML), and multiple myeloma.

In certain embodiments, the cancer for treatment with the compound is a lymphoma selected from Hodgkin's lymphoma, Non-Hodgkin's lymphoma, and Burkitt's lymphoma.

In certain embodiments, the cancer for treatment with the compound is a cancer characterized by mesenchymal features or mesenchymal phenotype. In some cancers, gain of mesenchymal features is associated with migratory (e.g., intravasation) and invasiveness of cancers. Mesenchymal features can include, among others, enhanced migratory capacity, invasiveness, elevated resistance to apoptosis, and increased production of extracellular matrix (ECM) components. In addition to these physiological characteristics, the mesenchymal features can include expression of certain biomarkers, including among others, E-cadherin, N-cadherin, integrins, FSP-1, α-SMA, vimentin, β-catenin, collagen I, collagen II, collagen III, collagen IV, fibronectin, laminin 5, SNAIL-1, SNAIL-2, Twist-1, Twist-2, and Lef-1. In certain embodiments, the cancer selected for treatment with the compounds herein include, among others, breast cancer, lung cancer, head and neck cancer, prostate cancer, and colon cancer. In certain embodiments, the mesenchymal features can be inherent to the cancer type or induced by or selected for by treatment of cancers with chemotherapy and/or radiation therapy.

In certain embodiments, the cancer for treatment with the compound is identified as having or determined to have an activating or oncogenic RAS activity. In certain embodiments, the RAS is K-RAS, H-RAS or N-RAS. In certain embodiments, the activating or oncogenic RAS is an activating or oncogenic RAS mutation.

In certain embodiments, the cancer selected for treatment with the compounds are determined to have or identified as having an activating or oncogenic RAS activity. In certain embodiments, the activating or oncogenic RAS activity is an activating or oncogenic RAS mutations. In certain embodiments, the activating or oncogenic RAS activity is an activating or activating K-RAS activity, particularly an activating or oncogenic K-RAS mutation. In certain embodiments, the activating or oncogenic RAS activity is an activating or activating N-RAS activity, particularly an activating or oncogenic N-RAS mutation. In certain embodiments, the activating or oncogenic RAS activity is an activating or activating H-RAS activity, particularly an activating or oncogenic H-RAS mutation.

In certain embodiments, the cancer for treatment with the compounds can be a cancer having prevalence (e.g., at least about 10% or more, or about 15% or more of the cancers), of an activating or oncogenic RAS mutation, such as biliary tract cancer, cervical cancer, endometrial cancer, pancreatic cancer, lung cancer, colorectal cancer, head and neck cancer, stomach (gastric) cancer, hematologic cancer (e.g., leukemia, lymphomas, etc.), ovarian cancer, prostate cancer, salivary gland cancer, skin cancer, small intestinal cancer, thyroid cancer, aerodigestive tract, urinary tract cancer, and bladder cancer.

In certain embodiments, the compounds can be used to treat a cancer that is refractory to one or more other chemotherapeutic agents, particularly cytotoxic chemotherapeutic agents; or treat a cancer resistant to radiation treatment. In certain embodiments, the compounds are used to treat cancers that have developed tolerance to chemotherapeutic agents activating other cell death pathways, such as apoptosis, mitotic catastrophe, necrosis, senescence and/or autophagy.

In certain embodiments, the cancer for treatment with the compounds is identified as being refractory or resistant to chemotherapy. In certain embodiments, the cancer is refractory or resistant to one or more of alkylating agents, anti-cancer antibiotic agents, antimetabolic agents (e.g., folate antagonists, purine analogs, pyrimidine analogs, etc.), topoisomerase inhibiting agents, anti-microtubule agents (e.g., taxanes, vinca alkaloids), hormonal agents (e.g., aromatase inhibitors), plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors.

In certain embodiments, the cancer for treatment with the compounds is a cancer identified as being refractory or resistant to one or more of afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelborae), vincristine, vinblastine, vinorelbine, and vindesine.

In certain embodiments, the cancer for treatment with the compound is identified as being refractory or resistant to one or more chemotherapeutics agents selected from cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, and erlotinib.

In certain embodiments, the cancer for treatment with the compounds is a cancer resistant to ionizing radiation therapy. The radioresistance of the cancer can be inherent or as a result of radiation therapy. In certain embodiments, the cancers for treatment with the compounds is, among others, a radioresistant adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemia and lymphoma), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, or vaginal cancer. In certain embodiments, the cancer is pancreatic cancer, breast cancer, glioblastoma, advanced non-small-cell lung cancer, bladder cancer, sarcoma, or soft tissue carcinoma.

### 4. Combination Treatments

In certain embodiments, the compounds described herein are used in combination with one or more of other (e.g., second therapeutic agent) therapeutic treatments for cancer. In certain embodiments, the compounds can be used as monotherapy, or as further provided below, in a combination therapy with one or more therapeutic treatments, particularly in combination with one or more chemotherapeutic agents. In certain embodiments, the compounds are used in combination with a second therapeutic agent, where the compounds are used at levels that sensitizes the cancer or cancer cell to the second therapeutic agent, for example at levels of the compound that do not cause significant cell death. In certain embodiments, the compounds can be used in combination with radiation therapy, either to sensitize the cells to radiation therapy or as an adjunct to radiation therapy (e.g., at doses sufficient to activate cell death pathway).

In certain embodiments, a subject with cancer is treated with a combination of a compound described herein and radiation therapy. In certain embodiments, the method comprises administering to a subject with cancer a therapeutically effective amount of a compound of the disclosure, and adjunctively treating the subject with an effective amount of radiation therapy. In certain embodiments, the compound is administered to the subject in need thereof prior to, concurrently with, or subsequent to the treatment with radiation.

In certain embodiments, the method comprises administering an effective amount of a compound described herein to a subject with cancer to sensitize the cancer to radiation treatment, and administering a therapeutically effective amount of radiation therapy to treat the cancer. In certain embodiments, an effective amount of X-ray and gamma ray is administered to the subject. In certain embodiments, an effective amount of particle radiation is administered to the subject, where the particle radiation is selected from electron beam, proton beam, and neutron beam radiation. In certain embodiments, the radiation therapy is fractionated.

In certain embodiments, a subject with cancer is administered a therapeutically effective amount of a compound described herein, or a first pharmaceutical composition thereof, and adjunctively administered a therapeutically effective amount of a second chemotherapeutic agent, or a second pharmaceutical composition thereof.

In certain embodiments, the second chemotherapeutic agent is selected from an platinating agent, alkylating agent, anti-cancer antibiotic agent, antimetabolic agent (e.g., folate antagonists, purine analogs, pyrimidine analogs, etc.), topoisomerase I inhibiting agent, topoisomerase II inhibiting agent antimicrotubule agent (e.g., taxanes, vinca alkaloids), hormonal agent (e.g., aromatase inhibitors), plant-derived agent and synthetic derivatives thereof, anti-angiogenic agent, differentiation inducing agent, cell growth arrest inducing agent, apoptosis inducing agent, cytotoxic agent, agent affecting cell bioenergetics, i.e., affecting cellular ATP levels and molecules/activities regulating these levels, anti-cancer biologic agent (e.g., monoclonal antibodies), kinase inhibitors and inhibitors of growth factors and their receptors.

In certain embodiments, the second chemotherapeutic agent is an angiogenesis inhibitor, such as but not limited to, an inhibitor of soluble VEGFR-1, NRP-1, angiopoietin 2, TSP-1, TSP-2, angiostatin and related molecules, endostatin, vasostatin, calreticulin, platelet factor-4, TIMP, CDAI, Meth-1, Meth-2, IFN-α, IFN-β, IFN-γ, CXCL10, IL-4, IL-12, IL-18, prothrombin (kringle domain-2), antithrombin III fragment, prolactin, VEGI, SPARC, osteopontin, maspin, canstatin (a fragment of COL4A2), or proliferin-related protein. In certain embodiments, the angiogenesis inhibitor is bevacizumab (Avastin), itraconazole, carboxyamidotriazole, TNP-470 (an analog of fumagillin), CM101, IFN-α, IL-12, platelet factor-4, suramin, SU5416, thrombospondin, a VEGFR antagonist, an angiostatic steroid plus heparin, cartilage-derived angiogenesis inhibitory factor (CDAI), a matrix metalloproteinase inhibitor, angiostatin, endostatin, 2-methoxyestradiol, tecogalan, tetrathiomolybdate, thalidomide, thrombospondin, prolactin, a αVβ3 inhibitor, linomide, ramucirumab, tasquinimod, ranibizumab, sorafenib (Nexavar), sunitinib (Sutent), pazopanib (Votrient), or everolimus (Afinitor).

In certain embodiments, the second chemotherapeutic agent is a cyclin-dependent kinase (CDK) inhibitor (e.g., a CDK4/CDK6 inhibitor). Examples include, but are not limited to, palbociclib (Ibrance), Ribociclib (optionally further in combination with letrozole), abemaciclib (LY2835219; Verzenio), P1446A-05, and Trilaciclib (G1T28).

In certain embodiments, the second chemotherapeutic agent is a Bruton's tyrosine kinase (BTK) inhibitor, such as but not limited to, Ibrutinib (PCI-32765), acalabrutinib, ONO-4059 (GS-4059), spebrutinib (AVL-292, CC-292), BGB-3111, and HM71224.

In certain embodiments, the second chemotherapeutic agent is a BRAF inhibitor. Examples include, but are not limited to, BAY43-9006 (Sorafenib, Nexavar), PLX-4032 (Vemurafenib), GDC-0879, PLX-4720, dabrafenib and LGX818.

In certain embodiments, the second chemotherapeutic agent is a EGFR inhibitor. Examples include, but are not limited to, gefitinib, erlotinib, afatinib, brigatinib, icotinib, cetuximab, osimertinib, panitumumab, brigatinib, lapatinib, cimaVax-EGF, and veristrat.

In certain embodiments, the second chemotherapeutic agent is a human epidermal growth factor receptor 2 (HER2) inhibitor. Examples include, but are not limited to, trastuzumab, pertuzumab (optionally further in combination with trastuzumab), margetuximab, and NeuVax.

In certain embodiments, disclosed herein is a method of increasing a subject's responsiveness to an immunotherapeutic or immunogenic chemotherapeutic agent, the method comprising administering to the subject in need thereof an effective amount of a compound described herein and an effective amount of an immunotherapeutic agent and/or an immunogenic chemotherapeutic agent. In certain embodiments, the method further includes administering to the subject a lipoxygenase inhibitor. In certain embodiments, the subject has a tumor whose cellular microenvironment is stromal cell rich. In certain embodiments, the administration of compound described herein results in killing one or more stromal cells in the tumor cells' microenvironment. In certain embodiments, the administration of an effective amount of an immunotherapeutic agent and/or an immunogenic chemotherapeutic agent results in killing one or more tumor cells. Also provided herein is a combination comprising a compound described herein and an immunotherapeutic agent, lipoxygenase inhibitor, or immunogenic chemotherapeutic agent. In certain embodiments, the immunotherapeutic agent is selected from a CTLA4, PDL1 or PD1 inhibitor. In certain embodiments, the immunotherapeutic agent can be selected from CTLA4 inhibitor such as ipilimumab, a PD1 inhibitor such as pembrolizumab or nivolumab or a PDL1 inhibitor such as atezolizumab or durvalumab. In certain embodiments, the immunotherapeutic agent is pembrolizumab. In other embodiments, the immunogenic chemotherapeutic agent is a compound selected from anthracycline, doxorubicin, cyclophosphamide, paclitaxel, docetaxel, cisplatin, oxaliplatin or carboplatin. In certain embodiments, provided herein is a combination comprising a compound described herein and a lipoxygenase inhibitor. In certain embodiments, the lipoxygenase inhibitor is selected from PD147176 and/or ML351. In certain embodiments, the lipoxygenase inhibitor may be a 15-lipoxygenase inhibitor (see, e.g., Sadeghian et al., Expert Opinion on Therapeutic Patents, 2015, 26:1, 65-88).

In certain embodiments, the second chemotherapeutic agent is selected from an alkylating agent, including, but not limiting to, adozelesin, altretamine, bendamustine, bizelesin, busulfan, carboplatin, carboquone, carmofur, carmustine, chlorambucil, cisplatin, cyclophosphamide, dacarbazine, estramustine, etoglucid, fotemustine, hepsulfam, ifosfamide, improsulfan, irofulven, lomustine, mannosulfan, mechlorethamine, melphalan, mitobronitol, nedaplatin, nimustine, oxaliplatin, piposulfan, prednimustine, procarbazine, ranimustine, satraplatin, semustine, streptozocin, temozolomide, thiotepa, treosulfan, triaziquone, triethylenemelamine, triplatin tetranitrate, trofosphamide, and uramustine; an antibiotic, including, but not limiting to, aclarubicin, amrubicin, bleomycin, dactinomycin, daunorubicin, doxorubicin, elsamitrucin, epirubicin, idarubicin, menogaril, mitomycin, neocarzinostatin, pentostatin, pirarubicin, plicamycin, valrubicin, and zorubicin; an antimetabolite, including, but not limiting to, aminopterin, azacitidine, azathioprine, capecitabine, cladribine, clofarabine, cytarabine, decitabine, floxuridine, fludarabine, 5-fluorouracil, gemcitabine, hydroxyurea, mercaptopurine, methotrexate, nelarabine, pemetrexed, raltitrexed, tegafur-uracil, thioguanine, trimethoprim, trimetrexate, and vidarabine; an immunotherapy, an antibody therapy, including, but not limiting to, alemtuzumab, bevacizumab, cetuximab, galiximab, gemtuzumab, panitumumab, pertuzumab, rituximab, brentuximab, tositumomab, trastuzumab, 90 Y ibritumomab tiuxetan, ipilimumab, tremelimumab and anti-CTLA-4 antibodies; a hormone or hormone antagonist, including, but not limiting to, anastrozole, androgens, buserelin, diethylstilbestrol, exemestane, flutamide, fulvestrant, goserelin, idoxifene, letrozole, leuprolide, magestrol, raloxifene, tamoxifen, and toremifene; a taxane, including, but not limiting to, DJ-927, docetaxel, TPI 287, larotaxel, ortataxel, paclitaxel, DHA-paclitaxel, and tesetaxel; a retinoid, including, but not limiting to, alitretinoin, bexarotene, fenretinide, isotretinoin, and tretinoin; an alkaloid, including, but not limiting to, demecolcine, homoharringtonine, vinblastine, vincristine, vindesine, vinflunine, and vinorelbine; an antiangiogenic agent, including, but not limiting to, AE-941 (GW786034, Neovastat), ABT-510, 2-methoxyestradiol, lenalidomide, and thalidomide; a topoisomerase inhibitor, including, but not limiting to, amsacrine, belotecan, edotecarin, etoposide, etoposide phosphate, exatecan, irinotecan (also active metabolite SN-38 (7-ethyl-10-hydroxy-camptothecin)), lucanthone, mitoxantrone, pixantrone, rubitecan, teniposide, topotecan, and 9-aminocamptothecin; a kinase inhibitor, including, but not liming to, axitinib (AG 013736), dasatinib (BMS 354825), erlotinib, gefitinib, flavopiridol, imatinib mesylate, lapatinib, motesanib diphosphate (AMG 706), nilotinib (AMN107), seliciclib, sorafenib, sunitinib malate, AEE-788, BMS-599626, UCN-01 (7-hydroxystaurosporine), vemurafenib, dabrafenib, selumetinib, paradox breakers (such as PLX8394 or PLX7904), LGX818, BGB-283, pexidartinib (PLX3397) and vatalanib; a targeted signal transduction inhibitor including, but not limiting to bortezomib, geldanamycin, and rapamycin; a biological response modifier, including, but not limiting to, imiquimod, interferon-α, and interleukin-2; and other chemotherapeutics, including, but not limiting to 3-AP (3-amino-2-carboxyaldehyde thiosemicarbazone), altrasentan, aminoglutethimide, anagrelide, asparaginase, bryostatin-1, cilengitide, elesclomol, eribulin mesylate (E7389), ixabepilone, lonidamine, masoprocol, mitoguanazone, oblimersen, sulindac, testolactone, tiazofurin, mTOR inhibitors (e.g. sirolimus, temsirolimus, everolimus, deforolimus, INK28, AZD8055, PI3K inhibitors (e.g. BEZ235, GDC-0941, XL147, XL765, BMK120), cyclin dependent kinase (CDK) inhibitors (e.g., a CDK4 inhibitor or a CDK6 inhibitor, such as Palbociclib (PD-0332991), Ribocyclib (LEE011), Abemaciclib (LY2835219), P1446A-05, Abemaciclib (LY2835219), Trilaciclib (G1T28), etc.), AKT inhibitors, Hsp90 inhibitors (e.g. geldanamycin, radicicol, tanespimycin), farnesyltransferase inhibitors (e.g. tipifarnib), Aromatase inhibitors (anastrozole letrozole exemestane); an MEK inhibitor including, but are not limited to, AS703026, AZD6244 (Selumetinib), AZD8330, BIX 02188, CI-1040 (PD184352), GSK1120212 (also known as trametinib or JTP-74057), cobimetinib, PD0325901, PD318088, PD98059, RDEA119 (BAY 869766), TAK-733 and U0126-EtOH; tyrosine kinase inhibitors, including, but are not limited to, AEE788, AG-1478 (Tyrphostin AG-1478), AG-490, Apatinib (YN968D1), AV-412, AV-951(Tivozanib), Axitinib, AZD8931, BIBF1120 (Vargatef), BIBW2992 (Afatinib), BMS794833, BMS-599626, Brivanib (BMS-540215), Brivanib alaninate (BMS-582664), Cediranib (AZD2171), Chrysophanic acid (Chrysophanol), Crenolanib (CP-868569), CUDC-101, CYC116, Dovitinib Dilactic acid (TK1258 Dilactic acid), E7080, Erlotinib Hydrochloride (Tarceva, CP-358774, OSI-774, NSC-718781), Foretinib (GSK1363089, XL880), Gefitinib (ZD-1839 or Iressa), Imatinib (Gleevec), Imatinib Mesylate, Ki8751, KRN 633, Lapatinib (Tykerb), Linifanib (ABT-869), Masitinib (Masivet, AB1010), MGCD-265, Motesanib (AMG-706), MP-470, Mubritinib (TAK 165), Neratinib (HKI-272), NVP-BHG712, OSI-420 (Desmethyl Erlotinib, CP-473420), OSI-930, Pazopanib HCl, PD-153035 HCl, PD173074, Pelitinib (EKB-569), PF299804, Ponatinib (AP24534), PP121, RAF265 (CHIR-265), Raf265 derivative, Regorafenib (BAY 73-4506), Sorafenib Tosylate (Nexavar), Sunitinib Malate (Sutent), Telatinib (BAY 57-9352), TSU-68 (SU6668), Vandetanib (Zactima), Vatalanib dihydrochloride (PTK787), WZ3146, WZ4002, WZ8040, quizartinib, Cabozantinib, XL647, EGFR siRNA, FLT4 siRNA, KDR siRNA, Antidiabetic agents such as metformin, PPAR agonists (rosiglitazone, pioglitazone, bezafibrate, ciprofibrate, clofibrate, gemfibrozil, fenofibrate, indeglitazar), DPP4 inhibitors (sitagliptin, vildagliptin, saxagliptin, dutogliptin, gemigliptin, alogliptin) or an EGFR inhibitor, including, but not limited to, AEE-788, AP-26113, BIBW-2992 (Tovok), CI-1033, GW-572016, Iressa, LY2874455, RO-5323441, Tarceva (Erlotinib, OSI-774), CUDC-101 and WZ4002.

In certain embodiments, the second chemotherapeutic agent is selected from afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, idelalisib, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, olaparib, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelboraf), vincristine, vinblastine, vinorelbine, vindesine, and the like. In certain embodiments, the compounds herein are administered prior to, concurrently with, or subsequent to the treatment with the chemotherapeutic agent.

In certain embodiments, the method of treating a cancer comprises administering a therapeutically effective amount of a compound described herein and a therapeutically effective amount a biologic agent used to treat cancer. In certain embodiments, the biologic agent is selected from anti-BAFF (e.g., belimumab); anti-CCR4 (e.g., mogamulizumab); anti-CD19/CD3 (e.g., blinatumomab); anti-CD20 (e.g., obinutuzumab, rituximab, ibritumomab tiuxetan, ofatumumab, tositumomab); anti-CD22 (e.g., moxetumomab pasudotox); anti-CD30 (e.g., brentuximab vedotin); anti-CD33 (e.g., gemtuzumab); anti-CD37 (e.g., otlertuzumab); anti-CD38 (e.g., daratumumab); anti-CD52 (e.g., alemtuzumab); anti-CD56 (e.g., lorvotuzumab mertansine); anti-CD74 (e.g., milatuzumab); anti-CD105; anti-CD248 (TEM1) (e.g., ontuxizumab); anti-CTLA4 (e.g., tremelimumab, ipilimumab); anti-EGFL7 (e.g., parsatuzumab); anti-EGFR (HER1/ERBB1) (e.g., panitumumab, nimotuzumab, necitumumab, cetuximab, imgatuzumab, futuximab); anti-FZD7 (e.g., vantictumab); anti-HER2 (ERBB2/neu) (e.g., margetuximab, pertuzumab, ado-trastuzumab emtansine, trastuzumab); anti-HER3 (ERBB3); anti-HGF (e.g., rilotumumab, ficlatuzumab); anti-IGF-1R (e.g., ganitumab, figitumumab, cixutumumab, dalotuzumab); anti-IGF-2R; anti-KIR (e.g., lirilumab, onartuzumab); anti-MMP9; anti-PD-1 (e.g., nivolumab, pidilizumab, lambrolizumab); anti-PD-L1 (e.g. Atezolizumab); anti-PDGFRa (e.g., ramucirumab, tovetumab); anti-PD-L2; anti-PIGF (e.g., ziv-aflibercept); anti-RANKL (e.g., denosumab); anti-TNFRSF 9 (CD 137/4-1 BB) (e.g., urelumab); anti-TRAIL-RI/DR4, R2/D5 (e.g., dulanermin); anti-TRAIL-R1/D4 (e.g., mapatumumab); anti-TRAIL-R2/D5 (e.g., conatumumab, lexatumumab, apomab); anti-VEGFA (e.g., bevacizumab, ziv-aflibercept); anti-VEGFB (e.g., ziv-aflibercept); and anti-VEGFR2 (e.g., ramucirumab).

### 5. Formulations and Administration

In certain embodiments, the pharmaceutical compositions of the therapeutic agents can be formulated by standard techniques using one or more physiologically acceptable carriers or excipients. Suitable pharmaceutical carriers are described herein and in Remington: The Science and Practice of Pharmacy, 21^{st} Ed. (2005). The therapeutic compounds and their physiologically acceptable salts, hydrates and solvates can be formulated for administration by any suitable route, including, among others, topically, nasally, orally, parenterally, rectally or by inhalation. In certain embodiments, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets, capsules, and solutions can be administered orally, rectally or vaginally.

For oral administration, a pharmaceutical composition can take the form of, for example, a tablet or a capsule prepared by conventional means with a pharmaceutically acceptable excipient. Tablets and capsules comprising the active ingredient can be prepared together with excipients such as: (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethylene glycol; (c) binders, e.g., magnesium aluminum silicate, starch paste, gelatin, tragacanth, methylcellulose, sodium carboxymethylcellulose, polyvinylpyrrolidone and/or hydroxypropyl methylcellulose; (d) disintegrants, e.g., starches (including potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners. The compositions are prepared according to conventional mixing, granulating or coating methods.

In certain embodiments, the carrier is a cyclodextrin, such as to enhance solubility and/or bioavailability of the compounds herein. In certain embodiments, the cyclodextrin for use in the pharmaceutical compositions can be selected from α-cyclodextrin, β-cyclodextrin, γ-cyclodextrin, derivatives thereof, and combinations thereof. In certain embodiments, the cyclodextrin is selected from β-cyclodextrin, γ-cyclodextrin, derivatives thereof, and combinations thereof.

In certain embodiments, the compounds can be formulated with a cyclodextrin or derivative thereof selected from carboxyalkyl cyclodextrin, hydroxyalkyl cyclodextrin, sulfoalkylether cyclodextrin, and an alkyl cyclodextrin. In various embodiments, the alkyl group in the cyclodextrin is methyl, ethyl, propyl, butyl,

When used in a formulation with the compound of the present disclosure, the cyclodextrin can be present at about 0.1 w/v to about 30% w/v, about 0.1 w/v to about 20% w/v, about 0.5% w/v to about 10% w/v, or about 1% w/v to about 5% w/v. In certain embodiments, the cyclodextrin is present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 12% w/v, about 14% w/v, about 16% w/v, about 18% w/v, about 20% w/v, about 25% w/v, or about 30% w/v or more.

Tablets may be either film coated or enteric coated according to methods known in the art. Liquid preparations for oral administration can take the form of, for example, solutions, syrups, or suspensions, or they can be presented as a dry product for reconstitution with water or other suitable vehicle before use. Such liquid preparations can be prepared by conventional means with pharmaceutically acceptable carriers and additives, for example, suspending agents, e.g., sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

The compounds can be formulated for parenteral administration, for example by bolus injection or continuous infusion. Formulations for injection can be presented in unit dosage form, for example, in ampoules or in multi-dose containers, with an optionally added preservative. Injectable compositions can be aqueous isotonic solutions or suspensions. In certain embodiments for parenteral administration, the compounds can be prepared with a surfactant, such as Cremaphor, or lipophilic solvents, such as triglycerides or liposomes. The compositions may be sterilized and/or contain adjuvants, such as preserving, stabilizing, wetting or emulsifying agents, solution promoters, salts for regulating the osmotic pressure and/or buffers. Alternatively, the compound can be in powder form for reconstitution with a suitable vehicle, for example, sterile pyrogen-free water, before use. In addition, they may also contain other therapeutically effective substances.

For administration by inhalation, the compound may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a compound with a carrier. Preferred carriers include absorbable pharmacologically acceptable solvents to assist passage through the skin of the subject. For example, transdermal devices are in the form of a bandage or patch comprising a backing member, a reservoir containing the compound optionally with carriers, optionally a rate controlling barrier to deliver the compound to the skin of the host at a controlled and predetermined rate over a prolonged period of time, and a means to secure the device to the skin. Matrix transdermal formulations may also be used.

Suitable formulations for topical application, e.g., to the skin and eyes, are preferably aqueous solutions, ointments, creams or gels well-known in the art. The formulations may contain solubilizers, stabilizers, tonicity enhancing agents, buffers and preservatives.

In certain embodiments, the compound can also be formulated as a rectal composition, for example, suppositories or retention enemas, for example, containing conventional suppository bases, for example, cocoa butter or other glycerides, or gel forming agents, such as carbomers.

In certain embodiments, the compound can be formulated as a depot preparation. Such long-acting formulations can be administered by implantation (for example, subcutaneously or intramuscularly) or by intramuscular injection. The compound can be formulated with suitable polymeric or hydrophobic materials (for example as an emulsion in an acceptable oil), ion exchange resins, biodegradable polymers, or as sparingly soluble derivatives, for example, as a sparingly soluble salt.

The pharmaceutical compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

### 6. Effective Amount and Dosing

In certain embodiments, a pharmaceutical composition of the compound is administered to a subject, preferably a human, at a therapeutically effective dose to prevent, treat, or control a condition or disease as described herein. The pharmaceutical composition is administered to a subject in an amount sufficient to elicit an effective therapeutic response in the subject. An effective therapeutic response is a response that at least partially arrests or slows the symptoms or complications of the condition or disease. An amount adequate to accomplish this is defined as “therapeutically effective dose” or “therapeutically effective amount.” The dosage of compounds can take into consideration, among others, the species of warm-blooded animal (mammal), the body weight, age, condition being treated, the severity of the condition being treated, the form of administration, route of administration. The size of the dose also will be determined by the existence, nature, and extent of any adverse effects that accompany the administration of a particular therapeutic compound in a particular subject.

In certain embodiments, a suitable dosage of the compounds of the disclosure or a composition thereof is from about 1 ng/kg to about 1000 mg/kg, from 0.01 mg/kg to 900 mg/kg, 0.1 mg/kg to 800 mg/kg, from about 1 mg/kg to about 700 mg/kg, from about 2 mg/kg to about 500 mg/kg, from about 3 mg/kg to about 400 mg/kg, 4 mg/kg to about 300 mg/kg, or from about 5 mg/kg to about 200 mg/kg. In certain embodiments, the suitable dosages of the compound can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. In certain embodiments, the dose of the compound can be administered once per day or divided into subdoses and administered in multiple doses, e.g., twice, three times, or four times per day.

In certain embodiments, the compounds can be administered with one or more of a second compound, sequentially or concurrently, either by the same route or by different routes of administration. When administered sequentially, the time between administrations is selected to benefit, among others, the therapeutic efficacy and/or safety of the combination treatment. In certain embodiments, the compounds herein can be administered first followed by a second compound, or alternatively, the second compound administered first followed by the compounds of the present disclosure. By way of example and not limitation, the time between administrations is about 1 hr, about 2 hr, about 4 hr, about 6 hr, about 12 hr, about 16 hr or about 20 hr. In certain embodiments, the time between administrations is about 1, about 2, about 3, about 4, about 5, about 6, or about 7 more days. In certain embodiments, the time between administrations is about 1 week, 2 weeks, 3 weeks, or 4 weeks or more. In certain embodiments, the time between administrations is about 1 month or 2 months or more.

When administered concurrently, the compound can be administered separately at the same time as the second compound, by the same or different routes, or administered in a single composition by the same route. In certain embodiments, the amount and frequency of administration of the second compound can used standard dosages and standard administration frequencies used for the particular compound. See, e.g., Physicians' Desk Reference, 70th Ed., PDR Network, 2015; incorporated herein by reference.

In certain embodiments where the compounds of the present disclosure is administered in combination with a second compound, the dose of the second compound is administered at a therapeutically effective dose. In certain embodiments, a suitable dose can be from about 1 ng/kg to about 1000 mg/kg, from about 0.01 mg/kg to about 900 mg/kg, from about 0.1 mg/kg to about 800 mg/kg, from about 1 mg/kg to about 700 mg/kg, from about 2 mg/kg to about 500 mg/kg, from about 3 mg/kg to about 400 mg/kg, from about 4 mg/kg to about 300 mg/kg, or from about 5 mg/kg to about 200 mg/kg. In certain embodiments, the suitable dosages of the second compound can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. In certain embodiments, guidance for dosages of the second compound is provided in Physicians' Desk Reference, 70^{th} Ed, PDR Network (2015), incorporated herein by reference.

It to be understood that optimum dosages, toxicity, and therapeutic efficacy of such compounds may vary depending on the relative potency of individual compound and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. compounds or combinations thereof that exhibit large therapeutic indices are preferred. While certain agents that exhibit toxic side effects can be used, care should be used to design a delivery system that targets such agents to the site of affected tissue to minimize potential damage to normal cells and, thereby, reduce side effects.

The data obtained from, for example, cell culture assays and animal studies can be used to formulate a dosage range for use in humans. The dosage of such small molecule compounds lies preferably within a range of circulating concentrations that include the ED₅₀ with little or no toxicity. The dosage can vary within this range depending upon the dosage form employed and the route of administration. For any compounds used in the methods disclosed herein, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

### 7. Methods of Preparation

The following examples are provided to further illustrate the methods of the present disclosure, and the compounds and compositions for use in the methods. The examples described are illustrative only and are not intended to limit the scope of the invention(s) in any way. The disclosures of all articles and references mentioned in this application, including patents, are incorporated herein by reference in their entirety.

The compounds of the present disclosure can be synthesized in view of the guidance provided herein, incorporating known chemical reactions and related procedures such as separation and purification. Representative methods and procedures for preparation of the compounds in this disclosure are described below and in the Examples. Acronyms are abbreviations are used per convention which can be found in literature and scientific journals.

It is understood that the starting materials and reaction conditions may be varied, the sequence of the reactions altered, and additional steps employed to produce compounds encompassed by the present disclosure, as demonstrated by the following examples. General references for known chemical reactions useful for synthesizing the disclosed compounds are available (see, e.g., Smith and March, March's Advanced Organic Chemistry: Reactions, Mechanisms, and Structure, Fifth Edition, Wiley Interscience, 2001; or Carey and Sundberg, Advanced Organic Chemistry, Part B. Reaction and Synthesis; Fifth Edition, Springer, 2007; or Li, J. J. Name Reactions, A Collection of Detailed Mechanisms and Synthetic Applications; Fifth Edition, Springer, 2014).

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

General Synthesis

In certain embodiments, compounds disclosed herein can be according to the general schemes shown below. Compounds of Formula I can be prepared according to the general syntheses outlined below in Schemes 1 and 2, where suitable reagents can be purchased form commercial sources or synthesized via known methods or methods adapted from the examples provided herein. Exemplary processes are show below in Schemes 1 and 2 for the synthesis of a compound of Formula I.

For example, as shown in Scheme 1, compounds of Formula I may prepared by first providing the 1H-benzo[d]imidazol-2-amine core, and then attaching the desired substituents using suitable coupling conditions (e.g., Suzuki coupling, Mitsunobu reaction, alkylation, etc.). In Scheme 1, each of ring A, X, R¹, R², R³, R⁴, p, and q are independently as defined herein.

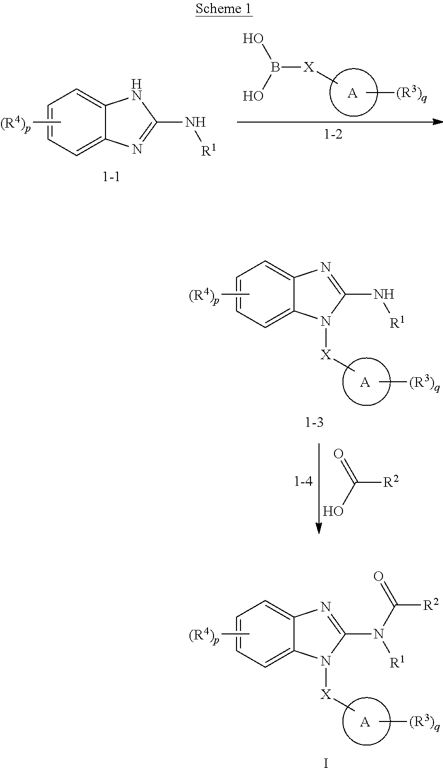

In Scheme 1, compound 1-3 can be provided by coupling amine 1-1 with boronic acid 1-2 under coupling conditions. Alternative cross coupling reactions can be employed as desired and thus alternative cross-coupling starting compounds, where compound 1-1 and 1-2 contain complimentary cross-coupling substituents. For example, derivatives of compound I-2 may be employed, where the boronic acid is a derivative thereof, such as a boronic ester, or a zinc or magnesium halide, an organotin compound, such as tributylstannane or trimethylstannane, and the like. The reaction is typically conducted in the presence of suitable catalyst such as a palladium catalyst including [1,1′-bis(diphenylphosphino)ferrocene]palladium(II) dichloride, Pd(PPh₃)₄, PdCl₂(PPh₃)₂ or tris(dibenzylideneacetone)dipalladium(0), and the like, or a copper catalyst such as CuCl, and if required suitable mediator, co-catalyst and/or base known to one skilled in the art using suitable solvents/solvent mixtures. Upon reaction completion, compound 1-3 can be recovered by conventional techniques such as neutralization, extraction, precipitation, chromatography, filtration and the like.

Referring to Scheme 2, compound 2-1 is coupled to compound 2-2 under standard coupling conditions to produce compound 2-3. The reaction is typically conducted in the presence of suitable catalyst (e.g., CuI) using suitable solvents/solvent mixtures. Hydrogenation of compound 2-3 provides compound 2-4. The 1H-benzo[d]imidazol-2-amine core of compound 2-5 can be formed by contacting compound 2-4 with cyanic bromide, whereafter further coupling and/or derivatization as described herein (e.g., Scheme 1) provides compounds of Formula I. Upon reaction completion, each intermediate can be recovered by conventional techniques such as neutralization, extraction, precipitation, chromatography, filtration and the like.

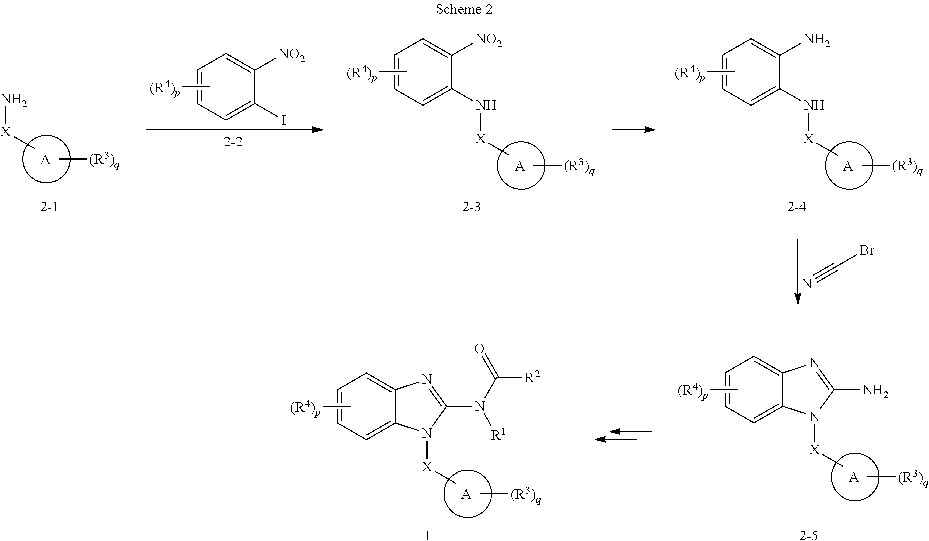

In some embodiments of the methods of Scheme 1 and Scheme 2, the various substituents on the starting compound (e.g., ring A, R¹, R², R³, etc.) are as defined for Formula I. However, it should also be appreciated that chemical derivatization and/or functional group interconversion, can be used to further modify of any of the compounds of Scheme 1 or Scheme 2 in order to provide the various compounds of Formula I. Appropriate starting materials and reagents can be purchased or prepared by methods known to one of skill in the art.

Compounds of Formula A-I can be prepared according to the general synthesis outlined below in Scheme A-1, where suitable reagents can be purchased form commercial sources or synthesized via known methods or methods adapted from the examples provided herein. Exemplary processes are shown below in Scheme A-1 the synthesis of a compound of Formula A-I. In Scheme A-1, each of R²¹, R², R³, R⁴, p, and q are independently as defined herein.

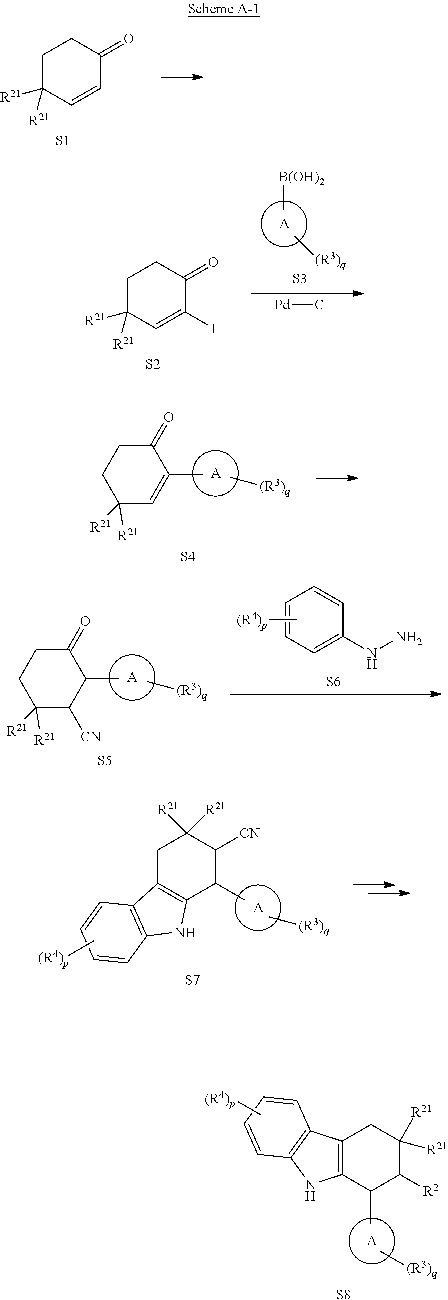

In Scheme A-1, halogenation of Si provides S2, which can then be coupled with compound S3 under standard palladium-catalyzed cross coupling conditions to provide S4. Cyanation of S4 yields S5, which conversion may utilize a transition metal catalyst (e.g., Cu). Cyclization of S5 with S6 provides tricycle S7. Further functional group interconversion can provide additional compounds of Formula A-I (e.g., S8). Upon each reaction completion, each of the intermediate or final compounds can be recovered, and optionally purified, by conventional techniques such as neutralization, extraction, precipitation, chromatography, filtration and the like.

Appropriate starting materials and reagents for use in Scheme A-1 can be purchased or prepared by methods known to one of skill in the art.

In some embodiments of the methods of Scheme A-1, the various substituents on the starting compounds (e.g., compounds S1, S3 and S6) are as defined for Formula A-I. However, it should also be appreciated that chemical derivatization and/or functional group interconversion, can be used to further modify of any of the compounds of Scheme A-1 in order to provide the various compounds of Formula A-I.

Other compounds of the disclosure can be synthesized using the synthetic routes above and adapting chemical synthetic procedures available to the skilled artisan. Exemplary methods of synthesis are provided in the Examples. It is to be understood that each of the procedures describing synthesis of exemplary compounds are part of the specification, and thus incorporated herein into the Detailed Description of this disclosure.

### SYNTHETIC EXAMPLES

### Example 1: Synthesis of Compound 1

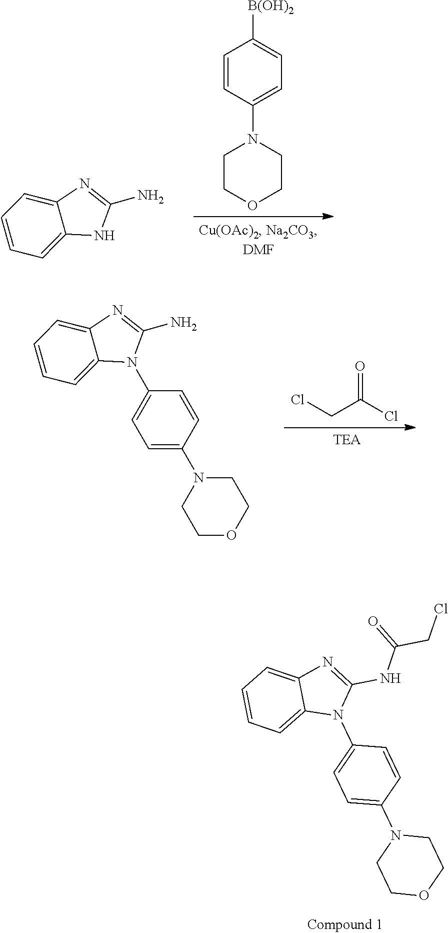 [SMILES: Nc1nc2ccccc2[nH]1 ; OB(O)c1ccc(N2CCOCC2)cc1 ; Nc1nc2ccccc2n1-c1ccc(N2CCOCC2)cc1 ; O=C(Cl)CCl ; O=C(CCl)Nc1nc2ccccc2n1-c1ccc(N2CCOCC2)cc1]

Preparation of 1-(4-morpholinophenyl)-1H-benzo[d]imidazol-2-amine: A mixture of 2-aminobenzimidazole (0.2 g, 1.49 mmol), (4-morpholinophenyl)boronic acid (0.39 g, 1.64 mmol), Cu(OAc)₂ (0.054 g, 0.29 mmol), Na₂CO₃ (0.31 mg, 2.98 mmol) and DMF (15 mL) was stirred at 60° C. for 12 h. The progress of the reaction was monitored by TLC and after completion of the reaction 10 ml of ice cold water was added to the reaction mixture and extracted two times with EtOAc (2×20 mL) and the combined organic phase was washed with sat. aq. NaHCO₃, dried over Na₂SO₄ and concentrated in vacuo. The crude product was purified via flash chromatography 5% MeOH in DCM to provide 1-(4-morpholinophenyl)-1H-benzo[d]imidazol-2-amine. LC-MS (m/z): 295.2 [M+H]⁺; ¹H NMR (400 MHz, DMSO): 3.19-3.27 (m, 4H), 3.72-3.76 (m, 4H), 6.14 (bs, 2H), 6.78-6.84 (m, 2H), 6.92-6.97 (m, 2H), 7.10-7.12 (m, 2H), 7.26-7.24 (m, 2H).

Preparation of 2-chloro-N-(1-(4-morpholinophenyl)-1H-benzo[d]imidazol-2-yl)acetamide: To a solution of 1-(4-morpholinophenyl)-1H-benzo[d]imidazol-2-amine (0.060 g, 0.20 mmol, 1 eq) in CH2Cl2 (10.0 mL) was added TEA (0.06 mL, 0.60 mmol, 3.0 eq) at 0° C., the reaction was stirred for 15 mins and then 2-chloroacetyl chloride (0.017 mL, 0.22 mmol, 1.5 eq) was added at 0° C. The reaction mixture was diluted with saturated NaHCO₃ solution (10 mL) and was extracted with DCM (2×50 mL). The organic layers were dried over Na₂SO₄ and concentrated to obtain the crude product. The crude product was purified by flash column chromatography using 30% EtOAc in hexane as an eluent to give 2-chloro-N-(1-(4-morpholinophenyl)-1H-benzo[d]imidazol-2-yl)acetamide. LC-MS (m/z): 371 [M+H]⁺; ¹H NMR (400 MHz, DMSO): 1.26 (s, 3H), 3.26 (m, 4H), 3.89 (s, 4H), 4.18 (s, 2H), 7.03-7.05 (m, 2H), 7.16-7.17 (m, 2H), 7.22-7.25 (m, 2H), 7.36-7.40 (m, 2H), 12.10 (bs, 1H).

### Example 2: Synthesis of Compound 2

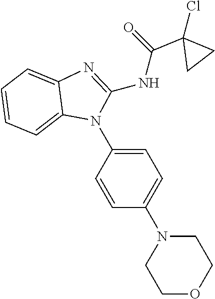 [SMILES: O=C(Nc1nc2ccccc2n1-c1ccc(N2CCOCC2)cc1)C1(Cl)CC1]

A similar synthetic scheme to Example 1 was used to synthesize Compound 2. LC-MS (m/z): 397.1 [M+H]⁺; ¹H NMR (400 MHz, DMSO-d₆): δ 1.18-1.24 (m, 2H), 1.41-1.48 (m, 2H), 3.19-3.22 (m, 4H), 3.74-3.75 (m, 4H), 7.08-7.09 (m, 3H), 7.14-7.23 (m, 2H), 7.36 (d, J=8.8 Hz, 2H), 7.51 (d, J=8 Hz, 1H), 12.63 (s, 1H).

### Example 3: Synthesis of Compound 3

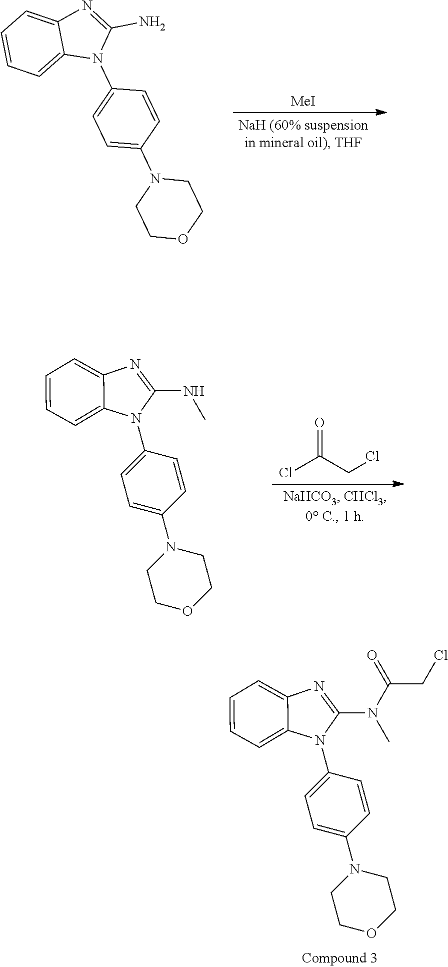 [SMILES: "MeI" ; Nc1nc2ccccc2n1-c1ccc(N2CCOCC2)cc1 ; CNc1nc2ccccc2n1-c1ccc(N2CCOCC2)cc1 ; O=C(Cl)CCl ; CN(C(=O)CCl)c1nc2ccccc2n1-c1ccc(N2CCOCC2)cc1]

Preparation of N-methyl-1-(4-morpholinophenyl)-1H-benzo[d]imidazol-2-amine: To a solution of 1-(4-morpholinophenyl)-1H-benzo[d]imidazol-2-amine (0.15 g, 0.50 mmol, 1 eq) in THF (15.0 mL) was added at −40° C. NaH (60%) (0.016 g, 0.40 mmol, 0.8 eq) and then the mixture was stirred at −40° C. for 0.5 h under N₂ atmosphere. TLC (5% MeOH in DCM) showed the reaction was completed. The reaction was warmed to room temperature and was diluted with ice water (15 mL), and extracted with EtOAc (2×30 mL). The organic layer was dried over anhydrous Na₂SO₄ and concentrated under reduced pressure to obtain the crude product. The crude product was purified by flash column chromatography using 2-3% MeOH in DCM as an eluent to give N-methyl-1-(4-morpholinophenyl)-1H-benzo[d]imidazol-2-amine. LCMS (ES) m/z=309.2 [M+H]⁺; ¹H NMR (400 MHz, DMSO-d6) δ ppm 2.82-2.83 (m, 3H), 3.19 (s, 4H), 3.75 (s, 4H), 6.04 (bs, 1H), 6.72-6.74 (m, 1H), 6.80-6.84 (m, 1H), 6.93-6.97 (m, 1H), 7.10-7.12 (m, 2H), 7.22-7.26 (m, 3H).

Preparation of 2-chloro-N-methyl-N-(1-(4-morpholinophenyl)-1H-benzo[d]imidazol-2-yl)acetamide: To a solution of N-methyl-1-(4-morpholinophenyl)-1H-benzo[d]imidazol-2-amine (0.04 g, 0.12 mmol, 1 eq) in CHCl₃ (15.0 mL) was added NaHCO₃ (0.032 g, 0.38 mmol, 3.0 eq) at 0° C., the reaction was stirred for 15 min and then 2-chloroacetyl chloride (0.014 mL, 0.19 mmol, 1.5 eq) was added at 0° C. The mixture was stirred at room temperature for 1 h under N₂ atmosphere. TLC (50% EtOAc in hexane) showed the reaction was completed. The reaction was quenched with ice and was extracted with DCM (100 mL). The organic layer was washed with saturated NaHCO₃ solution (10 mL) and water (10 mL), the layers were separated, the organic layer was dried over anhydrous Na₂SO₄, and concentrated under reduced pressure to obtain the crude product. The crude product was purified by flash column chromatography using 30-35% EtOAc in hexane as an eluent to afford 2-chloro-N-methyl-N-(1-(4-morpholinophenyl)-1H-benzo[d]imidazol-2-yl)acetamide. LCMS (ES) m/z=385 [M+H]⁺. ¹H NMR (400 MHz, DMSO-d6) δ ppm 2.93 (bs, 3H), 3.21 (s, 4H), 3.74 (s, 4H), 4.45 (s, 2H), 7.11-7.19 (m, 3H), 7.28-7.40 (m, 4H), 7.69-7.71 (m, 1H).

### Example 4: Synthesis of Compound 4

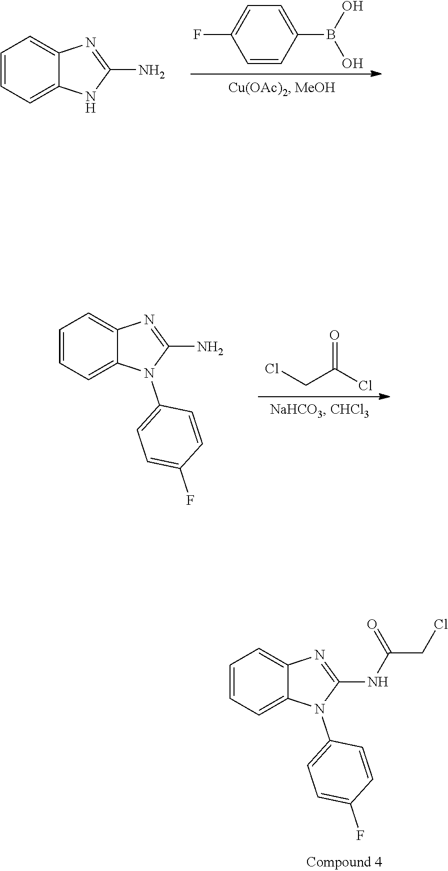 [SMILES: Nc1nc2ccccc2[nH]1 ; OB(O)c1ccc(F)cc1 ; Nc1nc2ccccc2n1-c1ccc(F)cc1 ; O=C(Cl)CCl ; O=C(CCl)Nc1nc2ccccc2n1-c1ccc(F)cc1]

Preparation of 1-(4-fluorophenyl)-1H-benzo[d]imidazol-2-amine: To a stirred mixture of 1H-benzo[d]imidazol-2-amine (0.3 g, 2.253 mmol, 1 eq), (4-fluorophenyl)boronic acid (0.37 g, 2.703 mmol, 1.2 eq) in methanol (10 mL) was added Cu(OAc)₂ (0.08 g, 0.450 mmol, 0.2 eq) at room temperature. The mixture was heated to 60° C. and stirred for 48 h. The progress of the reaction was monitored by TLC (10% methanol in dichloromethane). After completion of reaction, the mixture was filtered through Celite pad, the Celite pad was washed with methanol. The filtrate was concentrated under reduced pressure to obtain the crude product, which was purified by flash column chromatography using 6% methanol in dichloromethane as eluent to obtain 1-(4-fluorophenyl)-1H-benzo[d]imidazol-2-amine. LCMS (ES) m/z=228.1 [M+H]⁺; ¹H NMR (400 MHz, DMSO-d₆) δ ppm 6.24 (bs, 2H), 6.79-6.86 (m, 2H), 6.97 (t, J=7.2 Hz, 1H), 7.18-7.20 (m, 1H), 7.39-7.51 (m, 4H).

Preparation of 2-chloro-N-(1-(4-fluorophenyl)-1H-benzo[d]imidazol-2-yl)acetamide: To a stirred mixture of 1-(4-fluorophenyl)-1H-benzo[d]imidazol-2-amine (0.1 g, 0.44 mmol, 1 eq) and sodium bicarbonate (0.1 g, 1.32 mmol, 3.0 eq) in chloroform (10 mL), was added 2-chloroacetyl chloride (0.052 mL, 0.66 mmol, 1.5 eq) at 0° C. under nitrogen atmosphere. The resulting mixture was allowed to warm to room temperature and stirred for 2 h. The progress of the reaction was monitored by TLC (10% ethyl acetate in dichloromethane). After completion of the reaction, the mixture was diluted with dichloromethane (50 mL), washed with water (2×20 mL), dried over anhydrous sodium sulfate, filtered and concentrated under reduced pressure to obtain the crude product. The crude product was purified flash column chromatography using 4% ethyl acetate in dichloromethane as eluent to obtain 2-chloro-N-(1-(4-fluorophenyl)-1H-benzo[d]imidazol-2-yl)acetamide. LCMS (ES) m/z=304.2 [M+H]⁺; 1H NMR (400 MHz, DMSO-d6) δ ppm 4.13 (bs, 2H), 7.06 (s, 1H), 7.18-7.28 (m, 2H), 7.42 (t, J=8.0 Hz, 2H), 7.60 (bs, 3H), 12.82 (s, 1H).

### Example 5: Synthesis of Compound 5

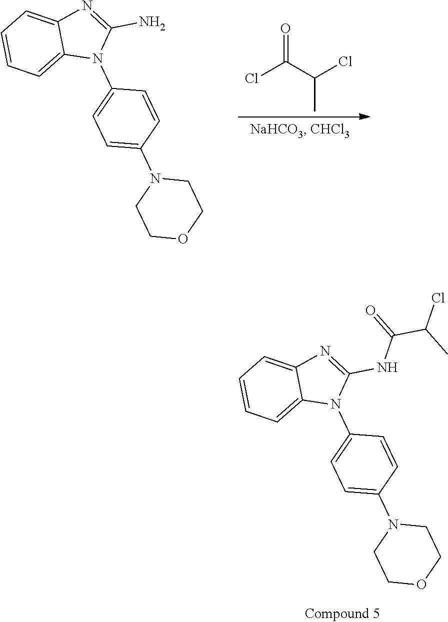

To a solution of 1-(4-morpholinophenyl)-1H-benzo[d]imidazol-2-amine (0.200 g, 0.68 mmol, 1.0 eq) in CHCl₃ (10.0 mL) was added NaHCO₃ (0.114 g, 1.35 mmol, 2.0 eq) and stirred for 15 min and 2-chloropropanoyl chloride (0.98 mL, 1.01 mmol, 1.5 eq) was added drop wise at 0° C. and stirred for 2.5 hours. LCMS and TLC (50% EtOAc in hexane) showed the reaction was completed. The reaction mixture was diluted with water (20 mL) and the organic layer was separated, dried over Na₂SO₄ and concentrated under reduced pressure to obtain the crude product. The crude product was purified by flash column chromatography using 20-30% EtOAc in hexane as an eluent to give 2-chloro-N-(1-(4-morpholinophenyl)-1H-benzo[d]imidazol-2-yl)propanamide. LC-MS (ES) m/z: 385.0 [M+H]⁺; ¹H NMR (400 MHz, DMSO-d₆) δ 1.53 (d, J=6.4 Hz, 3H), 3.20 (bs, 4H), 3.75 (bs, 4H), 4.41 (q, J=6.4 Hz, 1H), 7.04-7.11 (m, 3H), 7.17-7.24 (m, 2H), 7.26-7.36 (m, 2H), 7.38-7.56 (m, 1H), 12.37 (s, 1H).

### Example 6: Synthesis of Compound 6

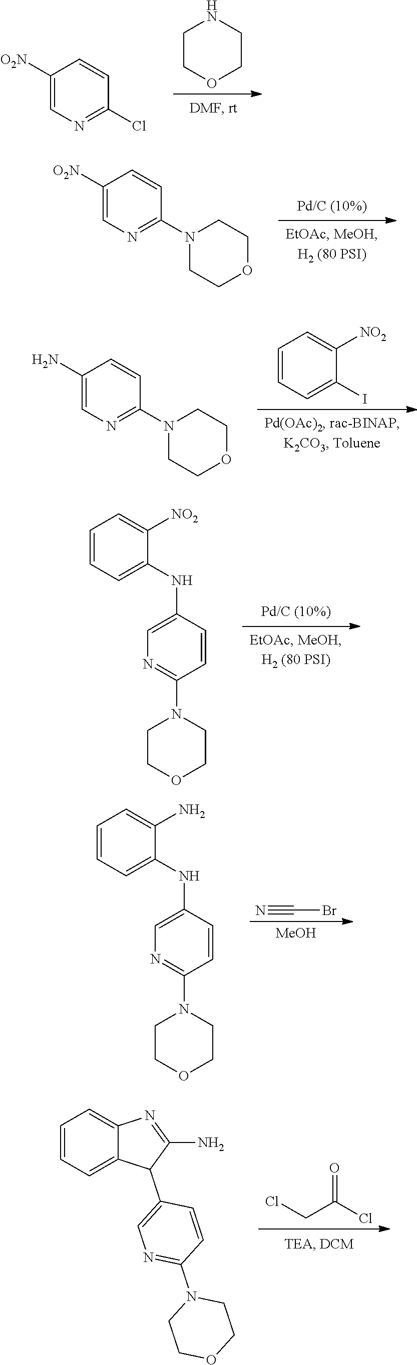

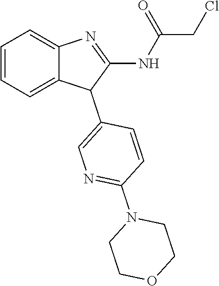 [SMILES: O=C(CCl)NC1=Nc2ccccc2C1c1ccc(N2CCOCC2)nc1]

A solution of 2-chloro-5-nitropyridine (3.0 g, 18.98 mmol, 1.0 eq) and morpholine (4.1 g, 47.46 mmol, 2.5 eq) in DMF (30 mL) was stirred at room temperature for 1 hour. After this time, the reaction mixture was diluted with water (50 mL) and precipitated solid was filtered to obtain 4-(5-nitropyridin-2-yl)morpholine. LCMS (ES) m/z=210.1 [M+H]⁺; ¹H NMR (400 MHz, DMSO-d₆) δ ppm 3.66-3.71 (m, 8H), 6.91 (d, J=9.2 Hz, 1H), 8.21 (dd, J=12.4, 2.8 Hz, 1H), 8.93 (d, J=5.6 Hz, 1H).

To a solution of 4-(5-nitropyridin-2-yl)morpholine (2.9 g, 13.87 mmol, 1.0 eq) in methanol (30 mL) and ethyl acetate (30 mL) was added Pd/C (0.2 g of 10 percent Pd) at room temperature. This reaction mixture was hydrogenated at 80 PSI in Parr shaker at room temperature for 8 h. After this time, the catalyst was removed by filtration through Celite bed, filtrate was concentrated under reduced pressure to obtain 6-morpholinopyridin-3-amine. LCMS (ES) m/z=180.1 [M+H]⁺; ¹H NMR (400 MHz, DMSO-d₆) δ ppm 3.15 (t, J=9.6 Hz, 4H), 3.65 (t, J=9.6 Hz, 4H), 4.55 (s, 2H), 6.60 (d, J=8.8 Hz, 1H), 6.90 (dd, J=8.8, 2.8 Hz, 1H), 7.59 (d, J=3.6 Hz, 1H).

A solution of 6-morpholinopyridin-3-amine (1.2 g, 6.70 mmol, 1.0 eq), 1-iodo-2-nitrobenzene (1.66 g, 6.70 mmol, 1.0 eq), Pd(OAC)₂ (45 mg, 0.20 mmol, 0.03 eq), rac-BINAP (0.208 g, 0.33 mmol, 0.05 eq), and K₂CO₃ (1.84 g, 13.40 mmol, 2.0 eq) in toluene (12 mL) was purged with N₂ for 10 minutes. The mixture was heated to 110° C. under N₂ for 16 h. After cooling to ambient temperature, the solvent was removed in vacuum and the crude product was purified by flash column chromatography on silica gel using EA/hex as the eluent to produce 6-morpholino-N-(2-nitrophenyl)pyridin-3-amine. LCMS (ES) m/z=301.1 [M+H]⁺; ¹H NMR (400 MHz, DMSO-d₆) δ ppm 3.43 (t, J=5.2 Hz, 4H), 3.69 (t, J=4.8 Hz, 4H), 6.52-6.89 (m, 3H), 7.42-7.48 (m, 1H), 7.52-7.54 (m, 1H), 8.07-8.09 (m, 2H), 9.27 (s, 1H).

To a solution of 6-morpholino-N-(2-nitrophenyl)pyridin-3-amine (1.6 g, 5.33 mmol, 1.0 eq) in methanol (25 mL) and ethyl acetate (25 mL) was added Pd/C (0.2 g of 10 percent Pd) at room temperature. This reaction mixture was hydrogenated at 80 PSI in Parr shaker at room temperature for 12 h. After this time, the catalyst was removed by filtration through Celite bed, filtrate was concentrated under reduced pressure and the crude product was purified by flash column chromatography on silica gel using MeOH/DCM as the eluent to produce N-(6-morpholinopyridin-3-yl)benzene-1,2-diamine. LCMS (ES) m/z=271.2 [M+H]⁺; ¹H NMR (400 MHz, CDCl₃) δ ppm 3.39 (t, J=4.4 Hz, 4H), 3.67 (bs, 2H), 3.83 (t, J=4.4 Hz, 4H), 4.94 (bs, 1H), 6.61 (d, J=8.8 Hz, 1H), 6.71-6.79 (m, 2H), 6.89-6.94 (m, 2H), 7.11-7.13 (m, 1H), 7.91 (d, J=2.0 Hz, 1H).

To a solution of 1-(6-morpholinopyridin-3-yl)benzene-1,2-diamine (0.4 g, 1.48 mmol, 1.0 eq) in methanol (7.0 mL) was added cyanic bromide (0.19 g, 1.77 mmol, 1.2 eq) at room temperature. The mixture was at room temperature for 2 h. After this time, the solvent was removed under reduced pressure and the crude product was purified by flash column chromatography on silica gel using MeOH/DCM as the eluent to produce 1-(6-morpholinopyridin-3-yl)-1H-benzo[d]imidazol-2-amine. LCMS (ES) m/z=296.2 [M+H]⁺; ¹H NMR (400 MHz, DMSO-d₆) δ ppm 3.54 (m, 4H), 3.71 (m, 4H), 6.19 (s, 2H), 6.72 (d, J=7.2 Hz, 1H), 6.81 (t, J=7.4 Hz, 1H), 6.93-7.01 (m, 2H), 7.16 (d, J=7.6 Hz, 1H), 7.61 (dd, J=8.8, 2.4 Hz, 1H), 8.16 (d, J=2.8 Hz, 1H).

To a solution of 1-(6-morpholinopyridin-3-yl)-1H-benzo[d]imidazol-2-amine (0.13 g, 0.44 mmol, 1 eq) in DCM (4.0 mL) was added triethyl amine (0.13 g, 1.32 mmol, 3.0 eq) at 0° C., followed by 2-chloroacetyl chloride (0.064 g, 0.57 mmol, 1.3 eq). The mixture was stirred at 0° C. for 1.0 h under N₂ atmosphere. TLC (5% MeOH in DCM) showed the reaction was completed. Then the reaction was diluted with saturated aqueous solution of NaHCO₃ (5 mL) and was extracted with DCM (25 mL). The organic layer was dried over anhydrous Na₂SO₄, filtered and concentrated under reduced pressure to obtain the crude product. The crude product was initially purified by preparative TLC using MeOH in DCM as mobile phase followed by preparative HPLC to give 2-chloro-N-(1-(6-morpholinopyridin-3-yl)-1H-benzo[d]imidazol-2-yl)acetamide. LCMS (ES) m/z=372.1 [M+H]⁺; ¹H NMR (400 MHz, DMSO-d₆) (at 60° C.) δ ppm 3.55-3.58 (m, 4H), 3.71-3.72 (m, 4H), 4.09 (s, 2H), 6.97 (d, J=8.8 Hz, 1H), 7.08 (bs, 1H), 7.18-7.27 (m, 2H), 7.58-7.69 (m, 2H), 8.25 (s, 1H), 12.70 (bs, 1H).

### Example 7: Synthesis of Compound 7

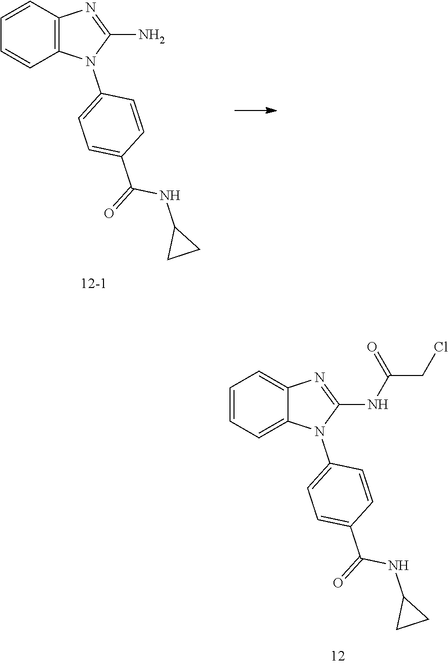 [SMILES: Nc1nc2ccccc2n1-c1ccc(C(=O)NC2CC2)cc1 ; O=C(CCl)Nc1nc2ccccc2n1-c1ccc(C(=O)NC2CC2)cc1]

A similar synthetic scheme to Example 8 was used to synthesize intermediate 12-1. LC-MS (m/z): 293.2 [M+H]⁺; H NMR (400 MHz, DMSO-d₆): δ 0.57 (s, 2H), 0.70 (d, J=5.2 Hz, 2H), 2.86-2.87 (m, 1H), 6.29 (s, 2H), 6.86 (d, J=3.2 Hz, 2H), 6.97-7.01 (m, 1H), 7.20 (d, J=8.0 Hz, 1H), 7.54 (d, J=8.4 Hz, 2H), 8.08 (d, J=8.4 Hz, 2H), 8.57 (d, J=3.6 Hz, 1H).

A similar synthetic scheme to Example 8 was used to synthesize Compound 12. LC-MS (m/z): 369.0 [M+H]⁺; ¹H NMR (400 MHz, DMSO-d₆): δ 0.58 (s, 2H), 0.70 (d, J=5.2 Hz, 2H), 2.86-2.87 (m, 1H), 4.14 (s, 2H), 7.14-7.29 (m, 3H), 7.58-7.65 (m, 3H), 7.99 (d, J=8.4 Hz, 2H), 8.58 (d, J=2.8 Hz, 1H), 12.80 (s, 0.5H).

### Example 8: Synthesis of Compound 8

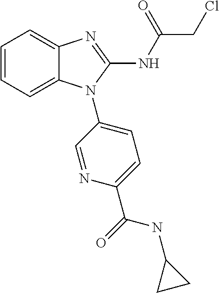 [SMILES: O=C(CCl)Nc1nc2ccccc2n1-c1ccc(C(=O)NC2CC2)nc1]

A similar synthetic scheme to Example 10 was used to synthesize Compound 13. LC-MS (m/z): 370.2 [M+H]⁺; ¹H NMR (400 MHz, DMSO-d₆): δ 0.68 (s, 4H), 2.92-2.94 (m, 1H), 4.14 (s, 2H), 7.22-7.29 (m, 3H), 7.58-7.59 (m, 1H), 8.19-8.22 (m, 2H), 8.87-8.88 (m, 2H), 12.9 (s, 1H).

Compounds as shown in Table 1, can be or were, synthesized according to the procedures described above using the appropriate reagents and starting materials.

### Example 9: Synthesis of Compounds A-13, A-14 and A-15

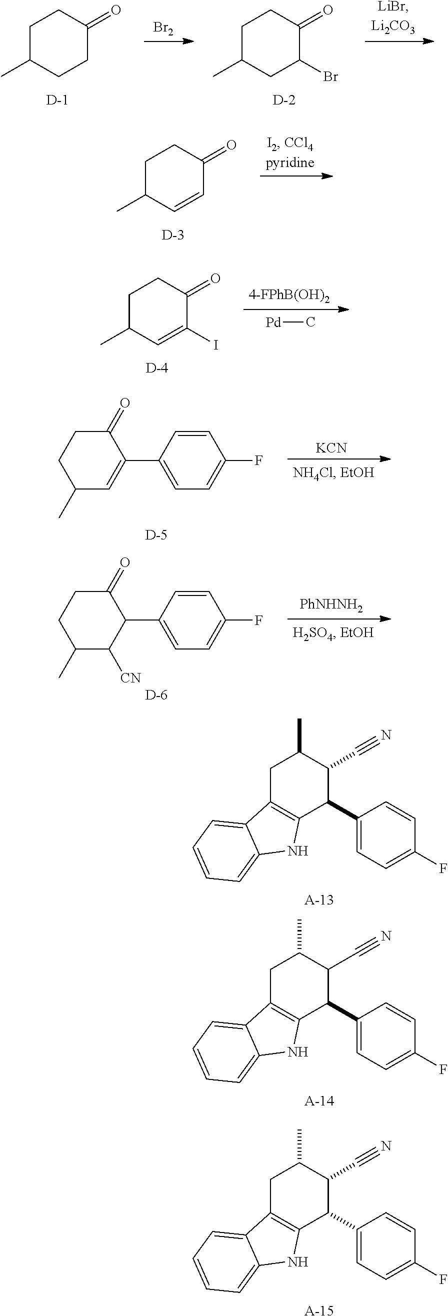

Preparation of Compound D-2 To a mixture of D-1 (10 g, 89.15 mmol, 10.94 mL, 1 eq) in water (30 mL) was added Br₂ (13.11 g, 82.02 mmol, 4.23 mL, 0.92 eq) dropwise. The mixture was stirred at 30° C. for 1.5 h to give a yellow mixture. TLC indicated the reaction was completed. The reaction mixture was diluted with 10 mL sat. Na₂S₂O₃ and 20 mL water, partitioned with 60 mL EtOAc and the layers separated. The aqueous layers was extracted with EtOAc (40 mL×3). All organic layer was washed twice with 30 mL sat. NaHCO₃, 30 mL brine and dried over anhydrous Na₂SO₄, filtered and concentrated in vacuum to give D-2 (15.6 g, crude).

Preparation of Compound D-3 To a mixture of D-2 (15.6 g, 81.65 mmol, 1 eq) and Li₂CO₃ (15.08 g, 204.12 mmol, 2.5 eq) in DMF (160 mL) was added LiBr (17.73 g, 204.12 mmol, 5.12 mL, 2.5 eq) in one portion. The mixture was stirred at 130° C. for 3 h under N₂ to give yellow mixture. TLC showed the reaction was completed. The reaction was diluted with 100 mL EtOAc and filtered. The filter cake was washed with EtOAc (30 mL 2). The resulting mixture was added water (300 mL) and the layers separated. The aqueous layers was extracted with EtOAc (80 mL×3). The combined organic layers were washed with water (200 mL×2) and brine (200 mL), dried over Na₂SO₄, filtered and concentrated to afford D-3 (15.2 g, crude).

Preparation of Compound D-4 A solution of Iodine (60.83 g, 239.67 mmol, 48.28 mL, 2 eq) in CCl4 (70 mL) was added dropwise to a solution of D-3 (13.2 g, 119.83 mmol, 1 eq) in CCl₄ (130 mL) and PYRIDINE (56.87 g, 719.00 mmol, 58.03 mL, 6 eq) at 0° C. The reaction mixture was allowed to warm to 25° C. and stirred for 16 hr to give a black brown solution. TLC indicated the reaction was completed. The mixture was diluted with MTBE (400 mL) and washed with the NaHCO₃ (aq., 800 mL). Na₂S₂O₃ (aq. 70 mL) was added dropwise to the mixture until the color of organic phase turns yellow. The aqueous phase was extracted with ethyl acetate (400 mL). The combined organic phase was washed with brine (600 mL×3), dried with anhydrous Na₂SO₄, filtered and concentrated in vacuum to give a residue. The residue was purified by column chromatography (SiO₂, Petroleum ether:Ethyl acetate=1:0 to 15:1) to afford D-4 (5.89 g, 24.95 mmol, 20.82% yield).

¹H NMR (400 MHz, CHLOROFORM-d) δ 1.19 (d, J=7.2 Hz, 3H), 1.70-1.83 (m, 1H), 2.10-2.23 (m, 1H), 2.50-2.59 (m, 1H), 2.63-2.72 (m, 1H), 2.73-2.82 (m, 1H), 7.60-7.62 (m, 1H)

Preparation of Compound D-5 To a solution of D-4 (5.89 g, 24.95 mmol, 1 eq) in DME (60 mL) and water (60 mL) were added Na₂CO₃ (5.29 g, 49.90 mmol, 2 eq), (4-fluorophenyl)boronic acid (6.98 g, 49.90 mmol, 2 eq) and Pd(dppf)Cl₂ (1.10 g, 1.50 mmol, 0.06 eq). The mixture was stirred at 25° C. for 12 hr to give black suspension. LCMS showed the starting material was consumed completely. The reaction was filtered. The filtrate was concentrated in reduced pressure to give a residue. The residue was purified by column chromatography (SiO₂, Petroleum ether:Ethyl acetate=1:0 to 20:1) to give D-5 (3.85 g, 18.85 mmol, 75.55% yield).

¹H NMR (400 MHz, CHLOROFORM-d) δ 1.17 (d, J=7.2 Hz, 3H), 1.62-1.76 (m, 1H), 2.08-2.14 (m, 1H), 2.39-2.51 (m, 1H), 2.53-2.62 (m, 1H), 2.62-2.71 (m, 1H), 6.74-6.77 (m, 1H), 6.90-7.00 (m, 2H), 7.17-7.24 (m, 2H)

Preparation of Compound D-6 To a solution of D-5 (1.5 g, 7.34 mmol, 1 eq) in EtOH (15 mL)/H₂O (15 mL) were added NH₄Cl (707.14 mg, 13.22 mmol, 1.8 eq) and cyanopotassium (956.46 mg, 14.69 mmol, 629.25 uL, 2 eq). The mixture was stirred at 100° C. for 3 h to give a yellow solution. TLC indicated the reaction was completed. The reaction mixture was basified to pH=8 with Sat.NaHCO₃. The mixture was concentrated to give the residue. The residue was extracted with EtOAc (50 mL×3). The organic layers was washed with brine (80 mL), dried with anhydrous Na₂SO₄, filtered and concentrated in vacuum to afford D-6 (1.8 g, crude).

General Procedure for Preparation of Compounds A-13, A-14, A-15

To a suspension of D-6 (200 mg, 864.81 μmol, 1 eq), phenylhydrazine (93.52 mg, 864.81 mol, 85.02 uL, 1 eq) in EtOH (7 mL) was added concentrated H₂SO₄ (169.64 mg, 1.73 mmol, 92.19 uL, 2 eq). The reaction mixture was stirred at 85° C. for 16 hr to give brown solution. LCMS showed starting material was consumed completely. After cooling, the reaction mixture was poured into ice water (50 ml). The aqueous phase was extracted with ethyl acetate (30 mL×4). The combined organic phase was washed with brine (50 mL×2), dried with anhydrous Na₂SO₄, filtered and concentrated in vacuum to give a residue. The residue was purified by prep-TLC (SiO₂, Petroleum ether:Ethyl acetate=3:1) to afford A-13 (1.07 mg, 3.52 μmol, 0.41% yield), A-14 (6.6 mg, 21.68 μmol, 2.51% yield) and A-15 (47.8 mg, 157.05 μmol, 18.16% yield)

Compound A-13: LC-MS (m/z): 304.9 [M+H]⁺

¹H NMR (400 MHz, CHLOROFORM-d) δ 1.37 (d, J=6.68 Hz, 3H), 2.23-2.35 (m, 1H), 2.42-2.53 (m, 1H), 2.60 (t, J=11.2 Hz, 1H), 2.97-3.07 (m, 1H), 4.27 (br d, J=10.0 Hz, 1H), 1H), 6.99-7.04 (m, 2H), 7.04-7.07 (m, 1H), 7.08-7.12 (m, 1H), 7.14-7.18 (m, 2H), 7.19-7.22 (m, 1H), 7.35 (br s, 1H), 7.43 (d, J=7.6 Hz, 1H).

Compound A-14: LC-MS (m/z): 305.1 [M+H]⁺

¹H NMR (400 MHz, CHLOROFORM-d) δ 1.27 (d, J=6.8 Hz, 3H), 2.36-2.49 (m, 1H), 2.69-2.81 (m, 1H), 2.97-3.01 (m, 1H), 3.02-3.09 (m, 1H), 4.48 (d, J=5.2 Hz, 1H), 6.99-7.07 (m, 2H), 7.09-7.23 (m, 4H), 7.27-7.31 (m, 1H), 7.52-7.57 (m, 1H), 7.60 (br s, 1H).

Compound A-15: LC-MS (m/z): 304.9 [M+H]⁺

¹H NMR (400 MHz, CHLOROFORM-d) δ 1.30 (d, J=6.8 Hz, 3H), 2.26-2.42 (m, 1H), 2.66-2.79 (m, 1H), 2.85-2.97 (m, 1H), 3.12-3.15 (m, 1H), 4.43 (br d, J=2.0 Hz, 1H), 7.00-7.08 (m, 3H), 7.10-7.13 (m, 1H), 7.17 (s, 1H), 7.24-7.32 (m, 2H), 7.46 (br d, J=6.8 Hz, 2H).

### Example 10: Synthesis of Compounds A-16, A-17 and A-18

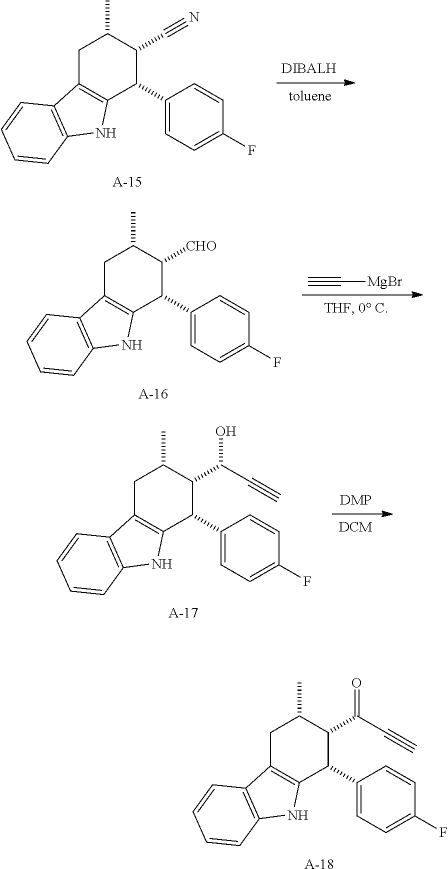

To a mixture of A-15 (220.00 mg, 722.83 μmol, 1 eq) in toluene (5 mL) was added DIBALH (1 M, 1.45 mL, 2 eq) in one portion at 0° C. under N₂. The mixture was stirred at 0° C. for 1 h to give brown mixture. TLC showed the reaction was completed. The reaction mixture was acidified to pH=3 with 2 mol/L HCl to give a brown mixture. The mixture was diluted with water (30 mL) and extracted with ethyl acetate (20 mL×3). The combined organic phase was washed with brine (30 mL×2), dried with anhydrous Na₂SO₄, filtered and concentrated in vacuum to afford A-16.

A solution of A-16 (80 mg, 260.28 μmol, 1 eq) in THF (5 mL) was added ethynylmagnesium bromide (0.5 M, 5.21 mL, 10 eq) under N₂. The reaction mixture was stirred at 0° C. for 1 h. TLC indicated the reaction was completed. The reaction mixture filtered and the filter was adjust to pH=6 by NH₄Cl aqueous solution. The resulting mixture was diluted with water (20 mL) and extracted with EtOAc (30 mL×3). The combined organic layers were washed with brine (30 mL), dried over anhydrous Na₂SO₄, filtered and concentrated under reduced pressure to give a residue. The residue was purified by prep-TLC (SiO₂, Petroleum ether:Ethyl acetate=4:1) to afford A-17.

To a mixture of A-17 (28 mg, 83.98 μmol, 1 eq) in DCM (3 mL) was added DMP (178.11 mg, 419.92 μmol, 130.00 uL, 5 eq) in one portion at 0° C. under N₂. The mixture was stirred at 0° C. for 2 h. LCMS indicated material was consumed completely. The reaction mixture was concentrated in reduced pressure to give a residue. The residue was dissolved with EtOAc (20 mL) and washed with water (20 mL). The aqueous phase was extracted with ethyl acetate (15 mL×2). The combined organic phase was washed with brine (30 mL), dried with anhydrous Na₂SO₄, filtered and concentrated in vacuo to give a residue. The residue was purified by prep-TLC (SiO₂, Petroleum ether:Ethyl acetate=5:1) to afford A-18. LC-MS (m/z): 331.9[M+H]⁺

¹H NMR (400 MHz, chloroform-d) δ 1.13 (d, J=6.0 Hz, 3H), 2.38-2.56 (m, 2H), 2.80 (t, J=10.4 Hz, 1H), 2.95-3.03 (m, 1H), 3.23 (s, 1H), 4.54 (br d, J=11.2 Hz, 1H), 6.91-6.98 (m, 2H), 7.03-7.11 (m, 4H), 7.13-7.16 (m, 1H), 7.30 (br s, 1H), 7.44 (d, J=6.8 Hz, 1H).

### Example 11: Synthesis of Compound A-19

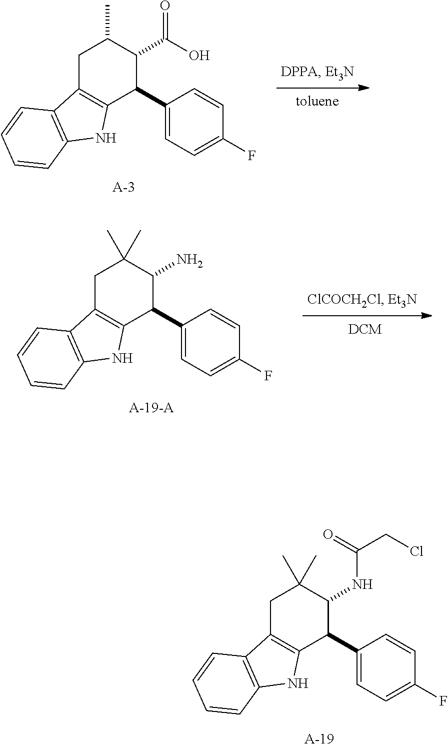

To a solution of A-3 (100 mg, 296.40 μmol, 1 eq) in toluene (5 mL) were added Et₃N (89.98 mg, 889.19 μmol, 123.76 uL, 3 eq) and DPPA (97.88 mg, 355.67 μmol, 77.07 uL, 1.2 eq). The mixture was stirred at 120° C. for 4 hr to give a yellow solution. LCMS and TLC (eluting with: PE/EtOAc=1/1) showed the reaction was completed. The reaction mixture was quenched with H₂O (10 mL). The mixture was stirred at 20° C. for 0.5 hr. The mixture was extracted with EtOAc (15 mL*3). The organic layers were dried over Na₂SO₄ and concentrated to give the crude product. The crude product was purified by flash column (eluting with: PE/EtOAc=100% PE to 40%) to give A-19-A.

To a solution of A-19-A (20 mg, 64.85 μmol, 1 eq) in DCM (5 mL)/H₂O (1 mL) were added NaHCO₃ (54.48 mg, 648.50 μmol, 25.22 uL, 10 eq) and 2-chloroacetyl chloride (21.97 mg, 194.55 μmol, 15.47 uL, 3 eq) at 0° C. The mixture was stirred at 0° C. for 0.5 hr to give a yellow suspension. LCMS showed the reaction was completed. The reaction mixture was diluted with H₂O (10 mL) and extracted with DCM (15 mL*3). The organic layers were dried over Na₂SO₄ and concentrated to give the crude product. The crude product was purified by prep-HPLC (column: Xtimate C18 150*25 mm*5 μm; mobile phase: [water (0.05% HCl)-ACN]; B %: 50%-80%, 8 min) to give A-19. LC-MS (m/z): 385.1 [M+H]⁺

¹H NMR (400 MHz, chloroform-d) δ ppm 0.98 (s, 3H) 1.08 (s, 3H) 2.68-2.72 (d, J=16.0 Hz, 1H) 2.82-2.86 (d, J=16.0 Hz, 1H) 3.71-3.75 (d, J=14.8 Hz, 1H) 3.90-3.94 (d, J=15.2 Hz, 1H) 4.24-4.29 (m, 1H) 6.42-6.45 (m, 1H) 6.94-7.11 (m, 7H) 7.43-7.45 (m, 2H).

### Example 12: Synthesis of A-20

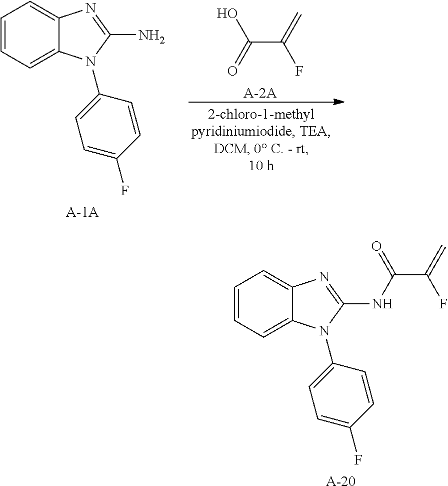

Preparation of 2-fluoro-N-[1-(4-fluorophenyl)-1H-1,3-benzodiazol-2-yl]prop-2-enamide (A-20) To a solution of 1-(4-fluorophenyl)-1H-1,3-benzodiazol-2-amine (0.2 g, 0.880 mmol, 1.0 equiv), 2-chloro-1-methyl-pyridinium iodide (0.27 g, 1.06 mmol, 1.2 equiv) and triethylamine (0.223 g, 2.20 mmol, 2.5 equiv) in DCM (5 mL), 2-fluoroprop-2-enoic acid (0.0809 g, 0.880 mmol, 1.0 equiv) was added at 0° C. The reaction mixture was stirred at 0° C. for 10 minutes. The reaction mixture was allowed to stir at RT for 10 h. The reaction mixture was quenched with ice water (50 mL), and extracted with DCM (2×50 mL). The combined organic layer was washed with brine (25 mL), dried over anhydrous sodium sulphate. The organic layer was filtered and concentrated under reduced pressure to get crude product, the crude product was purified by flash column chromatography using ethyl acetate in hexane. Product fractions were collected and concentrated under reduced pressure to get 2-fluoro-N-[1-(4-fluorophenyl)-1H-1,3-benzodiazol-2-yl]prop-2-enamide (A-20) (8 mg, 2.53%) as a white solid. LCMS (ES) m/z=300.3 [M+H]⁺.

¹H NMR (400 MHz, DMSO-d6) δ ppm: 5.12-5.15 (d, J=8 Hz, 1H), 5.39-5.51 (s, 1H), 7.12-7.25 (m, 411), 7.27-7.44 (m, 2H), 7.19-7.59 (m, 2H), 12.39 (s, 1H).

### Example 13: Synthesis of A-21

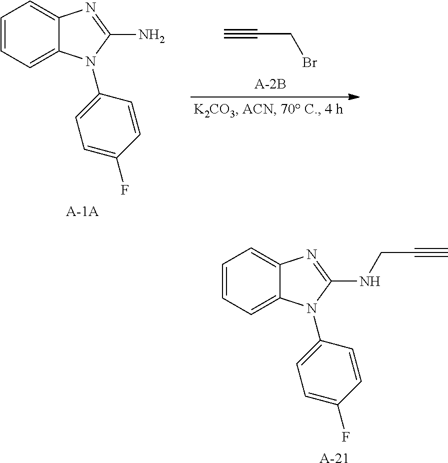

Preparation of 1-(4-fluorophenyl)-N-(prop-2-yn-1-yl)-1H-1,3-benzodiazol-2-amine (A-21) To a solution of 1-(4-fluorophenyl)-1H-1,3-benzodiazol-2-amine (0.5 g, 2.2 mmol, 1.0 equiv), and K₂CO₃ (0.912 g, 6.60 mmol, 3 equiv) in ACN (10 mL) at 0° C. was added 3-bromoprop-1-yne (0.164 g, 2.2 mmol, 0.5 equiv). The reaction mixture was stirred at 0° C. for 10 minutes. The reaction mixture was allowed to stir at 70° C. for 4 h. The reaction mixture was quenched with ice water (50 mL), extracted with ethyl acetate (2×30 mL). The combined organic layer was washed with brine (25 mL), dried over anhydrous sodium sulphate. Organic layer was filtered and concentrated under reduced pressure to get crude product. The crude was purified by flash column chromatography using ethyl acetate in hexane. Product fractions were collected and concentrated under reduced pressure to get 1-(4-fluorophenyl)-N-(prop-2-yn-1-yl)-1H-1,3-benzodiazol-2-amine (16 mg, 2.74%). LCMS (ES) m/z=316.3 [M+H]⁺.

¹H NMR (400 MHz, DMSO-d6) δ ppm 3.23-3.27 (d, J=16.0 Hz, 1H), 5.1 (s, 1H), 6.69 (s, 1H), 6.87-6.91 (m, 1H), 6.91-6.96 (m, 1H), 7.01-7.09 (m, 1H), 7.40 (s, 1H), 7.5 (s, 1H).

### Example 14 Synthesis of A-22

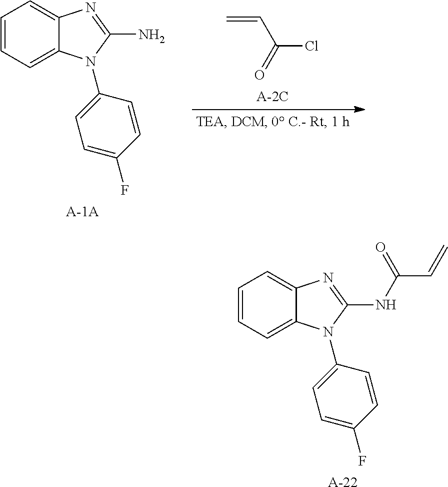

Preparation of compound N-[1-(4-fluorophenyl)-1H-1,3-benzodiazol-2-yl]prop-2-enamide (A-22). To a solution of 1-(44-fluorophenyl-1H-1,3-benzodiazol-2-amine (0.2 g, 0.880 mmol, 1.0 equiv) and triethylamine (0.267 g. 2.64 mmol, 3 equiv) in DCM (10 mL) at 0° C. was added prop-2-enoyl chloride and (95.6 mg, 1.06 mmol, 1.2 equiv). The reaction mixture was stirred at 0° C. for 10 minutes. The reaction mixture was allowed to stir at RT for 6 h. The reaction mixture was quenched with ice water (50 ml.), extracted with ethyl acetate (2×30 ml.). The combined organic layer was washed with brine (25 mL), dried over anhydrous sodium sulphate. Organic layer was filtered and concentrated under reduced pressure to get crude product. Obtained crude was purified by flash column chromatography using ethyl acetate in hexane. Product fractions were collected and concentrated under reduced pressure to get N-[1-(4-fluorophenyl)-1H-1,3-benzodiazol-2-yl] prop-2-enamide (35 mg; 14.14%). LCMS (ES) m/z=282.3 [M+H]⁺

¹H NMR (400 MHz, DMSO-d6) δ ppm: 5.8-5.9 (m, 1H), 6.12-6.15 (m, 2H), 7.25-7.33 (m, 3H), 7.34-7.38 (m, 2H), 7.50-7.61 (m, 3H), 10.39 (s, 1H).

The following compounds were synthesized using the procedures described above.

| | | | LCMS | |
|---|---|---|---|---|
| | | | m/z | |
| | Structure | Name | [M + H]⁺ | ¹H NMR (400 MHz, DMSO-d₆) |
| A-25 | 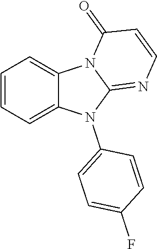 [SMILES: O=c1ccnc2n(-c3ccc(F)cc3)c3ccccc3n12] | 8-(4-fluorophenyl)-1,8,10- triazatricyclo[7.4.0.0²,⁷]trideca- 2,4,6,9,11-pentaen-13-one | 280.1 | 6.15 (d, J = 6.0 Hz, 1H), 7.31 (d, J = 8.0 Hz, 1H), 7.41-7.53 (m, 4H), 7.74-7.76 (m, 2H), 7.94 (d, J = 6.4 Hz, 1H), 8.58 (d, J = 7.6 Hz, 1H). |
| A-24 | 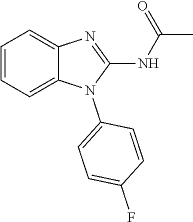 [SMILES: CC(=O)Nc1nc2ccccc2n1-c1ccc(F)cc1] | N-[1-(4-fluorophenyl)-1H-1,3- benzodiazol-2-yl]acetamide | 270.1 | 1.94 (s, 3H), 7.15-7.22 (m, 3H), 7.38-7.47 (m, 4H), 7.62 (s, 1H), 10.07 (s, 1H). (VT NMR at 90° C.) |
| A-23 | 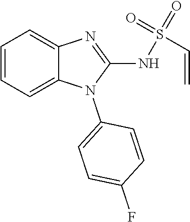 [SMILES: C=CS(=O)(=O)Nc1nc2ccccc2n1-c1ccc(F)cc1] | N-[1-(4-fluorophenyl)-1H-1,3- benzodiazol-2-yl]ethene-1- sulfonamide | 318.2 | 5.78 (d, J = 10 Hz, 1H), 6.10 (d, J = 16.4 Hz, 1H), 6.80-6.82 (m, 1H), 6.97 (d, J = 8.0 Hz, 1H), 7.16 (d, J = 8.4 Hz, 1H), 7.20-7.24 (m, 1H), 7.41-7.50 (m, 3H), 7.57-7.61 (m, 2H), 11.90 (s, 1H). |

Compounds of Formula A-I, or any compound disclosed herein, can be or were, synthesized according to the procedures described above using the appropriate reagents and starting materials.

### BIOLOGICAL EXAMPLES

### Biological Example 1: Cell Proliferation (Alamar Blue) Assay

Cell viability assay was performed to assess the potency of the compounds in human cancer cell lines 786-0 (renal cell carcinoma) and SJSA-1 (osteosarcoma). Additional cell lines, such as pancreatic cancer cell lines (Panc 02.13, BxPC-3, Panc 12, Panc 02.03, Panc 6.03, PSN-1, HPAC, and Capan-1), prostate cancer cell lines (PC-3, DU145, 22Rv1, NCI-H660, BPH1, LNCaP, BM-1604, and MDA PCa 2b), etc., can be tested in a similar method.

Cells (SJSA-1, 786-0 and A431) were seeded (5000 cells/100 μL/well) in 96-well tissue culture plate and incubated at 37° C./5% CO₂ for 16-24 hours. The cells were then treated with compounds (25 μL of 5×). The compound concentrations were 10-0.0005 μM prepared in 3-fold serial dilutions with final DMSO concentration of 1%. The plates were then incubated for 24 h at 37° C./5% CO₂ in a moist environment. Then Alamar Blue™ reagent (final concentration 1×-12.5 μL) was added to each well and incubated for 1.5 hours at 37° C./5% CO₂. The plates were read on fluorescence reader at 540 nm excitation and 590 nm emission wavelengths. The IC₅₀ values were subsequently determined using a sigmoidal dose-response curve (variable slope) in GraphPad Prism® 5 software. Table 2 shows cell proliferation data for exemplary compounds as described herein.

TABLE 2

| | IC₅₀ (μM) | | |
|---|---|---|---|
| No | 786-O | SJSA-1 | A431 |
| 1 | 0.007 | 0.012 | >10 |
| 2 | >10 | >10 | >10 |
| 3 | >10 | >10 | — |
| 4 | 1.8 | 1.7 | 4.4 |
| 5 | 0.049 | 0.015 | >10 |
| 6 | 0.001 | 0.003 | — |
| 7 | 0.060 | 0.083 | 8.2 |
| 8 | 0.023 | 0.032 | 6.7 |
| 9 | 0.0443 | 0.0663 | >10 |
| | 0.016 | 0.023 | |
| 10 | 0.494 | 0.651 | >10 |
| 12 | 0.189 | 0.366 | >10 |

Table 2-1 shows cell proliferation data for certain compounds that are inactive in cell proliferation assays described herein.

TABLE 2-1

| | IC₅₀ (μM) | | |
|---|---|---|---|
| Compound. | 786-O | SJSA-1 | A431 |
| 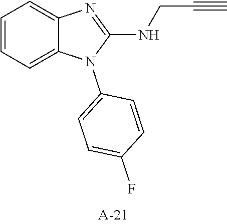 [SMILES: C#CCNc1nc2ccccc2n1-c1ccc(F)cc1] | >10 | NT | >10 |
| 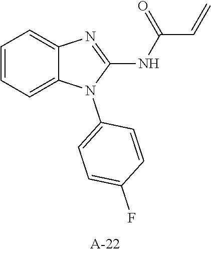 [SMILES: C=CC(=O)Nc1nc2ccccc2n1-c1ccc(F)cc1] | >10 | NT | >10 |
| 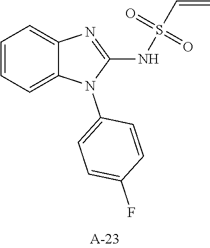 [SMILES: C=CS(=O)(=O)Nc1nc2ccccc2n1-c1ccc(F)cc1] | >10 | NT | >10 |
| 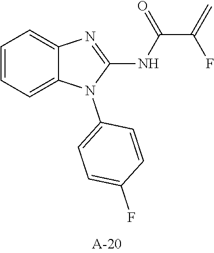 [SMILES: C=C(F)C(=O)Nc1nc2ccccc2n1-c1ccc(F)cc1] | >10 | NT | >10 |
| 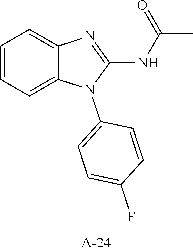 [SMILES: CC(=O)Nc1nc2ccccc2n1-c1ccc(F)cc1] | >10 | NT | >10 |

NT = not tested

### Biological Example 2: GPX4 Inhibition Assay

Table 5 shows that compounds provided herein are GPX4 inhibitors. Studies have shown that lipophilic antioxidants, such as ferrostatin, can rescue cells from GPX4 inhibition-induced ferroptosis. For instance, mesenchymal state GPX4-knockout cells can survive in the presence of ferrostatin, however, when the supply of ferrostatin is terminated, these cells undergo ferroptosis (see, e.g., Viswanathan et al., Nature 547:453-7, 2017). It has also been experimentally determined that that GPX4i can be rescued by blocking other components of the ferroptosis pathways, such as lipid ROS scavengers (Ferrostatin, Liproxstatin), lipoxygenase inhibitors, iron chelators and caspase inhibitors, which an apoptotic inhibitor does not rescue. These findings are suggestive of non-apoptotic, iron-dependent, oxidative cell death (i.e., ferroptosis). Accordingly, the ability of a molecule to induce ferroptotic cancer cell death, and that such ability is admonished by the addition of ferrostatin, is clear indication that the molecule is an GPX4 inhibitor. The data in Table 3 shows that compounds provided herein lost inhibitory activity in the presence of ferrostatin and are thus effective GPX4 inhibitors.

TABLE 3

| | 786-O (IC₅₀, μM) | | SJSA-1 (IC₅₀, μM) | |
|---|---|---|---|---|
| | Without | 2 μM | Without | 2 μM |
| No. | Ferrostatin | Ferrostatin | Ferrostatin | Ferrostatin |
| 13 | 0.028 | 7.171 | 0.022 | >10 |

### Biological Example 3: Method and Results of Western Blot—Gel Mobility Shift of GPX4

A mobility shift of GPX4 Western blot assay can be established to assess target engagement directly in cell-based assay after incubation with compounds and in tumors from mice treated with compounds. Mobility shift can be used as a pharmacodynamic marker for GPX4 irreversible inhibitors. For cell-based assay, cells that are sensitive to GPX4 inhibitors (e.g. MiaPaCa-2) are seeded in 10 cm (2-8×10⁶ cells) and grown overnight. Cell seeding number can be adjusted proportionally based on the surface area if smaller dishes are used. Next day, cells are treated with DMSO and various compounds at indicated concentrations for a period of time (e.g. 0.5, 1, 2, 4, 6, or up to 72 hours). Cells are then lysed in 0.3-0.5 mL of RIPA buffer (Sigma) supplemented with protease inhibitors (Roche) and phosphatase inhibitors (Sigma). Lysates are assayed for protein concentration using BCA kit (Pierce). Normalized amount of lysates (20-40 μg protein/lane) are run on 4-12% or 12% NuPage gel (Life Technologies) and the proteins are transferred to the PVDF or nitrocellulose membrane using iBlot® Transfer Stack (Life Technologies). The membranes are probed with primary antibodies shown in Table 4 at 4° C. overnight after blocking with 1×TBST containing 5% non-fat milk for one hour at room temperature. Similar antibodies from other vendors could also be used in Western blot analysis. After washing 5 times with 1×TBS containing 0.1% Tween20, the membranes were probed with 2^{nd} antibodies (e.g. Anti-mouse-HRP, Anti-rabbit-HRP, Anti-Goat-HRP, Anti-mouse IgG Dylight 800 conjugate or Anti-rabbit IgG DyLight 680 conjugate (1:10000; Cell signaling or similar IR 2^{nd} antibodies from different vendors) at room temperature for one hour. After washing 5 times, the membranes are scanned using ImageQuant-LAS-4010 (chemiluminiscence) (GE Healthcare) if HRP-conjugated secondary antibodies are used or Odyssey® Imaging System (Licor Biosciences) if infrared conjugated secondary antibodies are used.

TABLE 4
Primary antibodies used for Western blot analysis

| Antibody Name | Vendor | Cat No. | Species | MW | Dilution |
|---|---|---|---|---|---|
| β-Actin | Sigma | A5441 | Mouse | 43 kd | 1:10000 |
| (loading control) | | | | | |
| Vinculin | Sigma | V9131 | Mouse | 116 KD | 1:2000 |
| (loading control) | | | | | |
| GPX4 | Abcam | ab125066 | Rabbit | 22 kd | 1:1000 |
| GPX4 | Abcam | ab41787 | Rabbit | 22 kd | 1:1000 |

Compound can be evaluated in cell-based Western bot analysis of GPX4. In DMSO treated sample, GPX4 ran as doublet—the major lower free or unbound GPX4 band and the minor upper band (likely glutathione-bound GPX4 (Cozza et al., Free Radical Biology and Medicine, Vol 112, pages 1-11, 2017)). The amount of upper band can be reduced if samples were boiled in excess amount of reducing agent DTT. GPX4 in SDS-PAGE reducing gel moved slower (appear as a larger molecular weight protein) when treated with covalent, irreversible inhibitors of GPX4 (e.g. RSL-3 and ML162) but not reversible inhibitors (e.g. ML210), presumably due to addition of the covalently linked small molecule to GPX4. Unlike glutathione-bound GPX4, the irreversible inhibitor bound GPX4 upper band can't be reduced by excess amount of DTT. Further, distance of the GPX4 mobility shift is correlated with the molecular weight of the irreversible GPX4 inhibitor—shifted distance is bigger with larger irreversible inhibitors. Thus, this simple mobility shift of GPX4 Western blot can be used to conveniently assess direct target engagement in vitro, in cells and in tumors by irreversible inhibitors. It is contemplated that treatment of MiaPaCa-2 cells with compounds disclosed herein result in dose-dependent mobility shift of GPX4 from the lower unbound to upper bound bands.

### Biological Example 4: Kinact/Ki Determination for GPX4 Inhibitors

Day 1—seed cells: Cells are seeded with 5×10⁵ Calu6 cells/well into 5×6-well plates.

Day 2—treat cells with Cmpd, prepare samples for gels: Cells are treated with 1, 0.75, 0.5, 0.25 and 0.1 μM inhibitor+2 μM Ferrostatin-1 for 0, 10, 20, 30, 45, 60 minutes. 10 μL of 1000×DMSO stock solutions are prepared for each compound dilution (1, 0.75, 0.5, 0.25, 0.1 mM). Complete cell culture media (EMEM+10% FBS) is prepared with 2 μM Ferrostatin-1 final conc. Drug solutions are prepared by adding 1000× inhibitors to Ferrostatin-1-supplemented media at 1× final concentration (1, 0.75, 0.5, 0.25, 0.1 μM) plus DMSO for use as a negative control.

Cell lysis buffer is prepared by diluting 5× cell lysis buffer (Cell Signaling Technology #9803) and 100× protease/phosphatase inhibitor cocktail (Cell Signaling Technology #5872) to 1× with DI water.

Cells are treated with drug solutions in 1-hour time course. One concentration of drug added to each 6-well plate at t=60, 45, 30, 20, 10, 0 minutes. Media is aspirated from cells in 1 well of each 6-well plate and add 1 mL of media w/drug+ferrostatin (t=60 min). Cells are returned to incubator between time points. Media is aspirated and drug added to cells at each subsequent time point. At t=10 min DMSO is added negative control to additional well.

At t=0 media is aspirated from cells, cells are washed with ice cold PBS and aspirated, 75 μL of 1× cell lysis buffer is added per well, bottom of plates scraped with cell scraper, and lysates transferred to 1.5 mL Eppendorf tubes at store at −20° C.

SDS-PAGE running buffer is prepared (2 L of 1×MES Bolt running buffer (ThermoFisher Scientific #B0002), and stored at 4 C overnight for use the next day).

Day 3—perform BCA assay and run gels: Lysates are thawed on ice, centrifuged at 18,000×g at 4 C for 10 minutes, and BCA assay is performed on supernatant following manufacturer protocol (ThermoFisher Scientific #23225). 3.6×LDS/BME sample buffer is prepared by mixing Bolt 4×LDS sample buffer (ThermoFisher Scientific #B0008) with 2-mercaptoethanol at a 10:1 ratio. In 96-well PCR plate 19 μL 3.6×LDS/BME sample buffer is added and 50 μL lysate samples. Lysates diluted to 1 mg/mL with 1×LDS/BME, plates heated at 95 C for 10 min in PCR machine, loaded 15 uL/well (15 ug total lysate) into 12% Bis-Tris Bolt gels, and gels run at 200V for ˜35 minutes (until dye front reaches bottom of gel) with cold 1×MES running buffer. After which time, gels are washed 5 minutes in water, 10 minutes in 20% Ethanol/water, and transferred to membrane with iBlot2 (ThermoFisher Scientific). Membrane was blocked 1 h at RT with Licor TBS blocking buffer (Licor #927-60001) and incubated with 1:1000 dilution of anti-GPX4 antibody (Abcam #abl25066) in Licor TBS blocking buffer at 4 C overnight with gentle rocking.

Day 4—develop blots, quantify gel shift: Membrane is washed with 1×TBST for 30 minutes (change wash buffer 3-4 times), incubated with Licor secondary antibody (Licor #926-68021) 1:40,000 in Licor TBS blocking buffer for 1 h at RT with gentle rocking, washed with 1×TBST for 30 minutes, scraped with Licor imager and bands quantied with Image studio.

### Biological Example 5: Pharmacokinetics Studies

Male Balb/c mice (˜6-8 weeks old with body weight range of 22-25 g) and male SD rats (6-8 weeks old with body weight range of 200-250 g) can be procured from Vivo Biotech, Hyderabad, India. Animals are quarantined in for a period of 7 days with a 12:12 h light: dark cycles, and prior to the study the animals stratified as per body weight.

Housing: The animals are group housed in standard polycarbonate cages, with stainless steel top grill where pelleted food and drinking water bottle are placed; corn cob used as bedding material and changed at least twice a week or as required.

Diet ad libitum: Rodents feed manufactured by Altromin Spezialfutter GmbH & Co. KG., ImSeelenkamp20. D-32791 Lage, is provided.

Water ad libitum: Purified water is provided ad libitum to animals in polycarbonate bottles with stainless steel sipper tubes.

A) Procedure for Mice: Intravenous, oral and intraperitoneal pharmacokinetics study is done at doses of 5, 20 and 10 mg/kg respectively at dose volume of 10 mL/Kg for PO and IP while 5 mL/kg for IV route. Sparse sampling is done and at each time point three mice were used for blood sampling (˜100 μL) are collected from retro-orbital plexus at 0.083 (Only for IV), 0.25, 0.5, 1, 2, 4, 8, 10 (only for PO) and 24 h. Blood samples collected in tubes containing K₂.EDTA as anticoagulant and centrifuged for 5 min at 10,000 rpm in a refrigerated centrifuge (Biofuge, Heraeus, Germany) maintained at 4° C. for plasma separation.

Group I (IV) receive test compound intravenously by tail vein at 5 mg/Kg in solution formulation prepared using 30% Kolliphore EL in WFI; dose volume: 5 mL/Kg; strength: 1 mg/mL.

Group II (PO) receive test compound by per oral route using oral gavage needle at 20 mg/Kg in solution formulation prepared using 30% Kolliphore EL in WFI; dose volume: 10 mL/Kg; strength: 2 mg/mL.

Group III (IP) receive test compound by intraperitoneal route at 10 mg/Kg in solution formulation prepared using 30% Kolliphore EL in WFI; dose volume: 10 mL/Kg; strength: 1 mg/mL.

B) Procedure for rat: Intravenous and oral pharmacokinetics study is done at a dose 2 and 10 mg/kg at dose volume of 2 and 10 mL/Kg. Serial blood sampling is done and at each time point (˜200 μL) are collected from retro-orbital plexus at 0.083 (Only for IV), 0.25, 0.5, 1, 2, 4, 8, 10 (only for PO) and 24 h. Blood samples are collected in tubes containing K₂.EDTA as anticoagulant and centrifuged for 5 min at 10,000 rpm in a refrigerated centrifuge (Biofuge, Heraeus, Germany) maintained at 4° C. for plasma separation.

Group I (IV) receive test compound intravenously by tail vein at 2 mg/Kg in solution formulation prepared using 30% Kolliphore EL in WFI; dose volume: 2 mL/Kg; strength: 1 mg/mL.

Group II (PO) receive test compound using oral gavage needle at 10 mg/Kg (solution formulation prepared using 30% Kolliphore EL in WFI; dose volume: 10 mL/Kg: strength: 1 mg/mL.

Blood concentration-time data of test compound is analyzed by non-compartmental method using Phoenix WinNonlin Version 8.1.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A compound of Formula I, or a tautomer, stereoisomer, mixture of stereoisomers, isotopically enriched analog, or pharmaceutically acceptable salt thereof: 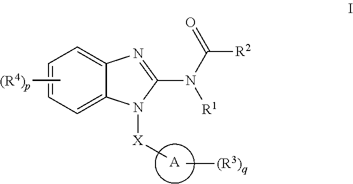
wherein:
ring A is C₄-C₁₀cycloalkyl, heterocyclyl, aryl, or heteroaryl;
X is a covalent bond or —C(R⁹)₂—;
p is 0, 1, 2 or 3;
q is 0, 1, 2 or 3;
R¹ is hydrogen or C₁-C₆alkyl;
R² is —C₁-C₂haloalkyl optionally substituted with one or two —CH₃;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₁₀alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;
each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₁₀alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;
each R⁹ is independently hydrogen or C₁-C₆alkyl;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl or C₃-C₁₀cycloalkyl;
each R¹³ is independently C₁-C₆alkyl or C₃-C₁₀cycloalkyl; and
each R¹¹ is independently C₁-C₆alkyl, C₂-C₆alkenyl, aryl, heteroaryl, —C₁-C₆alkylaryl, —C₂-C₆alkenylaryl, —C₁-C₆alkylheteroaryl, or —C₂-C₆alkenylheteroaryl.

2. The compound of claim 1, wherein ring A is C₄-C₁₀cycloalkyl.

3. The compound of claim 1, wherein ring A is heterocyclyl.

4. The compound of claim 1, wherein ring A is aryl.

5. The compound of claim 1, wherein ring A is heteroaryl.

6. The compound of claim 1, represented by a compound of Formula IIA, or a tautomer, stereoisomer, mixture of stereoisomers, isotopically enriched analog, or pharmaceutically acceptable salt thereof:

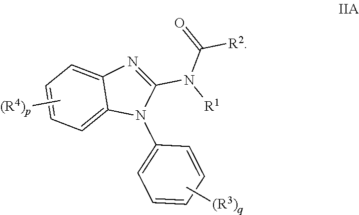

7. The compound of claim 1, represented by a compound of Formula IIIA, or a tautomer, stereoisomer, mixture of stereoisomers, isotopically enriched analog, or pharmaceutically acceptable salt thereof:

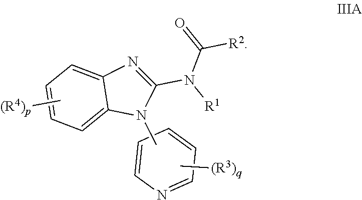

8. The compound of claim 1, represented by a compound of Formula IIB, or a tautomer, stereoisomer, mixture of stereoisomers, isotopically enriched analog, or pharmaceutically acceptable salt thereof: 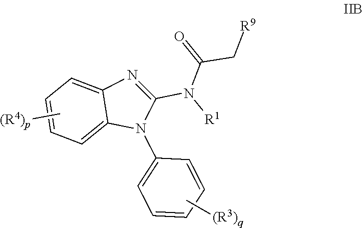
wherein R⁹ is halo.

9. The compound of claim 1, represented by a compound of Formula IIIB, or a tautomer, stereoisomer, mixture of stereoisomers, isotopically enriched analog, or pharmaceutically acceptable salt thereof: 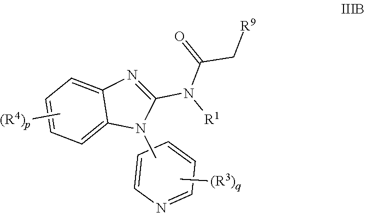
wherein R⁹ is halo.

10. The compound of any one of claims 1-9, wherein each R³ is independently halo, —C(O)N(R⁷)₂, or heterocyclyl.

11. The compound of any one of claims 1-9, wherein q is 1, and R³ is —S(O)₂N(R⁷)₂, —S(O)N(R⁷)₂, or —C(O)N(R⁷)₂.

12. The compound of any one of claims 1-9, wherein q is 1, and R³ is halo.

13. The compound of any one of claims 1-9, wherein q is 1, and R³ is —C(O)N(R⁷)₂.

14. The compound of any one of claims 1-9, wherein q is 1, and R³ is heterocyclyl.

15. The compound of any one of claims 1-9, wherein q is 0.

16. The compound of any one of claims 1-9, wherein p is 1, 2 or 3.

17. The compound of any one of claims 1-9, wherein p is 1.

18. The compound of any one of claims 1-17, wherein each R⁴ is independently halo, —CN, —OR⁸, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

19. The compound of any one of claims 1-17, wherein p is 0.

20. The compound of claim 1, represented by a compound of Formula IIC, or a tautomer, stereoisomer, mixture of stereoisomers, isotopically enriched analog, or pharmaceutically acceptable salt thereof: 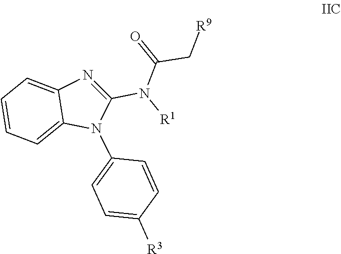
wherein R⁹ is halo.

21. The compound of claim 1, represented by a compound of Formula IIIC, or a tautomer, stereoisomer, mixture of stereoisomers, isotopically enriched analog, or pharmaceutically acceptable salt thereof: 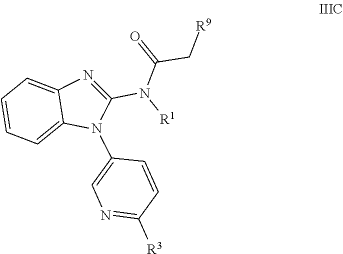
wherein R⁹ is halo.

22. The compound of any one of claims 1-21, wherein R¹ is C₁-C₆alkyl.

23. A compound of formula A-I: 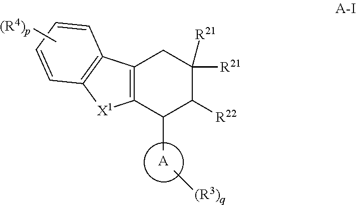
or a tautomer, stereoisomer, mixture of stereoisomers, isotopically enriched analog, or pharmaceutically acceptable salt thereof, wherein:
ring A is C₄-C₁₀cycloalkyl, heterocyclyl, aryl, or heteroaryl;
X¹ is NR⁵, O or S;
p is 0, 1, 2 or 3;
q is 0, 1, 2 or 3;
each R²¹ is independently hydrogen, C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R)₂, —NO₂, —OR⁸, —C₁-C₆alkyl-OH, —C₁-C₆alkyl-OR⁸, or —Si(R¹⁵)₃;
R²² is —CN, —C(O)H, —C(O)OH, ethyleneoxide, —C(O)-ethyleneoxide, —C(O)—C₁-C₂alkyl, —C(O)—C₁-C₂haloalkyl, —C(O)—C₂-C₃alkenyl, —C(O)—C₂alkynyl, —NHC(O)—C₁-C₂haloalkyl, —NHC(O)—C₂-C₃alkenyl, —NHC(O)—C₂alkynyl, —CH(OH)—C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;
each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;
R⁵ is hydrogen or C₁-C₆alkyl;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl or C₃-C₁₀cycloalkyl;
each R¹³ is independently C₁-C₆alkyl or C₃-C₁₀cycloalkyl; and
each R¹¹ is independently C₁-C₆alkyl, C₂-C₆alkenyl, aryl, heteroaryl, C₁-C₆alkylaryl, —C₂-C₆alkenylaryl, —C₁-C₆alkylheteroaryl, or —C₂-C₆alkenylheteroaryl.

24. The compound of claim 23, or a tautomer, stereoisomer, mixture of stereoisomers, isotopically enriched analog, or pharmaceutically acceptable salt thereof, represented by formula A-II:

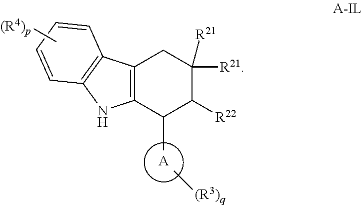

25. The compound of claim 23 or claim 24, wherein each R²¹ is independently C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸.

26. The compound of any one of claims 23-25, wherein each R²¹ is independently C₁-C₆alkyl, C₂-C₆alkenyl, C₂-C₆alkynyl, C₁-C₆haloalkyl, C₃-C₁₀cycloalkyl, —NH₂, —NHR⁸, —N(R⁸)₂, —OH, —OR⁸, —C₁-C₆alkyl-OH or —C₁-C₆alkyl-OR⁸.

27. The compound of any one of claims 23-26, wherein at least one R²¹ is C₁-C₆alkyl.

28. The compound of any one of claims 23-27, wherein each R²¹ is C₁-C₆alkyl.

29. The compound of any one of claims 23-28, wherein R²² is —C(O)—C₁-C₂alkylhalo.

30. The compound of any one of claims 23-29, wherein R²² is —CN.

31. The compound of any one of claims 23-30, wherein R²² is —C(O)C≡CH.

32. The compound of any one of claims 23-31, wherein X¹ is —NH—.

33. The compound of claim 23, or a tautomer, stereoisomer, mixture of stereoisomers, isotopically enriched analog, or pharmaceutically acceptable salt thereof, represented by formula A-III:

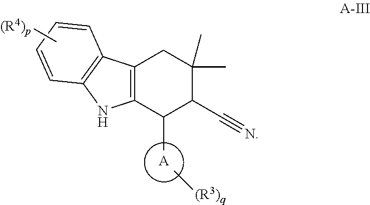

34. The compound of any one of claims 23-33, wherein each R⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently optionally substituted with one to three R¹⁰.

35. The compound of any one of claims 23-34, wherein each R⁴ is independently halo, —CN, —OH, —OR⁸, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

36. The compound of any one of claims 23-35, wherein ring A is C₄-C₁₀cycloalkyl.

37. The compound of any one of claims 23-36, wherein ring A is heterocyclyl.

38. The compound of any one of claims 23-37, wherein ring A is aryl.

39. The compound of any one of claims 23-38, wherein ring A is heteroaryl.

40. The compound of one of claims 23-39, wherein each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently optionally substituted with one to three R¹⁰.

41. The compound of any one of claims 23-40, wherein each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl is independently optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein
each R¹² is independently hydrogen, C₁-C₆alkyl or C₃-C₁₀cycloalkyl; and
each R¹³ is independently C₁-C₆alkyl or C₃-C₁₀cycloalkyl.

42. The compound of any one of claims 23-41, wherein at least one R³ is halo, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR, —OC(O)R⁸, —C(O)R⁶, or —OC(O)CHR⁸N(R¹²)₂.

43. The compound of any one of claims 23-42, wherein at least one R³ is halo.

44. The compound of any one of claims 23-43, wherein at least one R³ is —NHR⁸.

45. The compound of any one of claims 23-44, wherein at least one R³ is —C(O)OR⁶ or —C(O)R⁶.

46. The compound of any one of claims 23-45, wherein p is 0.

47. The compound of any one of claims 23-46, wherein p is 1, 2 or 3.

48. The compound of any one of claims 23-47, wherein p is 1.

49. The compound of any one of claims 23-48, wherein p is 2.

50. The compound of any one of claims 23-49, wherein q is 1.

51. The compound of any one of claims 23-50, wherein q is 2.

52. A compound selected from the group consisting of compounds listed in Table 1 or Table A-1, or a tautomer, stereoisomer, mixture of stereoisomers, isotopically enriched analog, or pharmaceutically acceptable salt thereof.

53. A pharmaceutical composition comprising a compound, or a tautomer, stereoisomer, mixture of stereoisomers, isotopically enriched analog, or pharmaceutically acceptable salt thereof, of any one of claims 1 to 52, and a pharmaceutically acceptable carrier.

54. A method of inhibiting GPX4 in a cell, comprising contacting a cell with an effective amount of a compound of any one of claims 1 to 52, or a tautomer, stereoisomer, mixture of stereoisomers, isotopically enriched analog, or pharmaceutically acceptable salt thereof, or a composition of claim 53.

55. The method of claim 54, wherein the cell is a cancer cell.

56. A method of treating cancer in a subject, comprising administering to a subject having cancer a therapeutically effective amount of a compound, or a tautomer, stereoisomer, mixture of stereoisomers, isotopically enriched analog, or pharmaceutically acceptable salt thereof, of any one of claims 1 to 52.

57. The method of claim 56, wherein the cancer is adrenocortical cancer, anal cancer, biliary cancer, bladder cancer, bone cancer, brain cancer, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, or a soft tissue carcinoma.

58. The method of claim 57, wherein the cancer is osteosarcoma, glioma, astrocytoma, neuroblastoma, cancer of the small intestine, bronchial cancer, small cell lung cancer, non-small cell lung cancer, basal cell carcinoma, or melanoma.

59. The method of claim 58, wherein the cancer is a the hematologic cancer.

60. The method of claim 59, wherein the hematologic cancer is acute lymphoblastic leukemia (ALL), acute myeloid leukemia (AML), lymphoma (e.g., Hodgkin's lymphoma, Non-Hodgkin's lymphoma, Burkitt's lymphoma), chronic lymphocytic leukemia (CLL), chronic myelogenous leukemia (CML), Hairy Cell chronic myelogenous leukemia (CML), or multiple myeloma.

61. The method of any one of claims 54 to 60, further comprising administering a therapeutically effective amount of a second therapeutic agent.

62. The method of claim 61, wherein the second therapeutic agent is a platinating agent, alkylating agent, anti-cancer antibiotic, antimetabolite, topoisomerase I inhibitor, topoisomerase II inhibitor, or antimicrotubule agent.
